(19) United States
(12) Patent Application Publication
(10) Pub. No.: US 2026/0331286 A1
(43) Pub. Date: Oct. 22, 2026

(54) Guideline, Prompt, and Response Conditioning in the Context of Generative Artificial Intelligence

(71) Applicant: Elevance Health, Inc., Indianapolis, IN (US)

(72) Inventors: Justin Vincent, Boston, MA (US); Madeline Nevermann, Milwaukee, WI (US)

(21) Appl. No.: 19/650,805

(22) Filed: Apr. 17, 2026

Related U.S. Application Data
(60) Provisional application No. 63/918,302, filed on Nov. 15, 2025.
(60) Provisional application No. 63/791,237, filed on Apr. 18, 2025.

(51) Int. Cl. G16H 50/20

(52) U.S. Cl. CPC G16H 50/20

(57) ABSTRACT

This application is directed to automatically generating prompts for reviewing user data files with reference to guideline documents using generative artificial intelligence. A computer system generates an ordered list of guideline policies based on a guideline document, and a structured data item based on a time series of user data files associated with a person. A medical request assessment application is executed to render a user interface on which the guideline policies and the structured data item are selected and presented. The computer system automatically generates prompts based on the ordered list of guideline policies and the structured data item, and applies a generative model to process the prompts and generate prompt results. Answers to the ordered list of guideline policies are determined based on the prompt results and displayed on the user interface, indicating whether the user data files of the person comply with the guideline policies.

### RELATED APPLICATIONS

This application claims benefits of U.S. Provisional Application No. 63/791,237, filed Apr. 18, 2025, titled “Guideline, Prompt, and Response Conditioning in the Context of Generative Artificial Intelligence” and U.S. Provisional Application No. 63/918,302, filed Nov. 15, 2025, titled “Secure Patient Health Information Management,” each of which is incorporated by reference in its entirety.

### TECHNICAL FIELD

This application relates generally to computer technology, and more particularly, to managing guidelines, prompts, and answers of generative artificial intelligence.

### BACKGROUND

Medical requests oftentimes undergo a thorough review process to ensure that a patient’s sensitive and often complex personal data, particularly information from their medical history, complies with the specific requirements outlined in long, detailed guideline handbooks. These handbooks typically contain comprehensive rules and regulations that need to be carefully followed, covering everything from eligibility for treatments to the use of certain medications. This review is carried out manually by medical professionals or administrative staff who sift through extensive medical records, cross-referencing various pieces of information to verify compliance. As the volume of medical data has grown and become more complex, various computer techniques have been gradually introduced over time to aid this process. Tools such as search functions, automated tagging, and basic data categorization have been implemented to help streamline the process by organizing medical records, highlighting key pieces of information, and making it easier for reviewers to find relevant data quickly. While these technologies have certainly provided some relief and have helped improve the efficiency of certain steps, the review process remains largely cumbersome, time-consuming, and prone to human error. Given these difficulties, there is a pressing need for a more efficient, automated solution—one that can intelligently assess medical requests with accuracy and precision while reducing the burden on reviewers.

While the advent of artificial intelligence (AI) offers the potential for automating some of these tasks, it is important to recognize that simply applying AI to process all the available data—without a targeted approach—may not necessarily result in an efficient solution. The sheer volume and complexity of medical histories, coupled with the specific nature of the guidelines, means that AI would need to be fine-tuned to assess only relevant aspects of the data, rather than being used as a “kitchen sink” solution to indiscriminately process everything. AI may either overlook important nuances or require extensive training to handle the full range of possible cases. This broad approach can lead to a tremendous waste of computation, storage, and communication resources of computer systems. An efficient and focused solution is required to utilize advanced computational techniques, such as machine learning and natural language processing, to autonomously analyze patient data, ensuring compliance with the guidelines in a more streamlined, accurate, and less resource-intensive manner. This would not only save time but also minimize errors, improve decision-making, and ultimately enhance the overall efficiency of utilizing computation, storage, and communication resources of computer systems.

Various embodiments of this application are directed to methods, system, and non-transitory computer-readable storage media for reviewing user data files with reference to guideline documents using generative artificial intelligence. A guideline document is translated to an ordered sequence of guideline policies (e.g., including one or more criteria and associated conditions), independently of any specific user data files. A time series of user data files associated with a person (e.g., a patient) are processed to generate a structured data item. The ordered sequence of guideline policies are applied to generate a set of prompts that may be customized based on the user data files for the person. The set of prompts are sent to a generative model to determine prompt results, which are further processed to provide user-readable answers to each of the ordered sequence of guideline policies. The answers are presented to a user requesting review of the user data files jointly with the guideline policies and/or the user data files. In some embodiments, the set of prompts and associated prompt results are generated automatically and without user intervention, and may be managed on the background without being visible to the user requesting review of the user data files. The set of prompts may be tuned to include important nuances to handle different cases. An efficient and focused solution is provided to utilize advanced computational techniques, such as machine learning and natural language processing, to autonomously analyze user data files (e.g., medical records) in compliance with guideline policies in a targeted, streamlined, accurate, and resource-friendly manner.

In one aspect, some implementations of this application are directed to a method implemented at a computing system for reviewing user data files. The method includes obtaining a guideline document for assessing a type of medical requests and generating an ordered list of guideline policies including one or more criteria and a plurality of conditions associated with each criterion based on the guideline document. Each guideline policy corresponds to at least a respective condition of a respective criterion. The method further includes obtaining a time series of user data files each of which includes unstructured data of a person and retrieving a plurality of information items from the unstructured data of each user data file to generate a structured data item associated with the time series of user data files. The method further includes executing a medical request assessment application, including rendering a graphical user interface, and receiving, on the graphical user interface, one or more user inputs of information identifying the guideline document and the user data files. The method further includes, in response to the one or more user inputs, displaying the ordered list of guideline policies associated with the guideline document on the graphical user interface; automatically and without user intervention, generating a plurality of first prompts based on the ordered list of guideline policies and the structured data item of the user data files, each guideline policy corresponding to a set of one or more respective first prompts; applying a generative model to generate a plurality of prompt results in response to the plurality of first prompts based on the user data files and the structure data item; and determining a plurality of answers to the ordered list of guideline policies. Each answer corresponds to a respective guideline policy and is determined based on prompt results of the set of one or more respective first prompts, indicating whether the time series of user data files comply with the respective guideline policy. The method further includes updating display of the ordered list of guideline policies based on the plurality of answers to the ordered list of guideline policies.

In another aspect, some implementations of this application are directed to a computer system including one or more processors and memory having instructions stored thereon. When executed by the one or more processors, the instructions cause the processors to perform operations for any of the above methods described herein.

In yet another aspect, some implementations of this application are directed to a non-transitory computer-readable storage medium, having instructions stored thereon, which when executed by one or more processors of a server system cause the processors to perform operations of any of the above methods described herein.

Generative artificial intelligence is applied in healthcare to process and analyze vast amounts of patient information with reference to complicated guideline polices, which is far beyond the capability of human management. The complexity and sheer volume of data involved — including medical histories, diagnostic images, lab results, and treatment plans — make it nearly impossible for humans to handle efficiently without a significant risk of error or delay. Computers, however, are uniquely equipped to manage and process this information at scale, utilizing artificial intelligence to swiftly analyze, correlate, and generate insights from the data. Unlike humans, computers can work tirelessly around the clock, handling multiple policies simultaneously and ensuring consistent, accurate outcomes. The ability to automate such processes through generative artificial intelligence not only reduces the burden on healthcare professionals but also significantly enhances the efficiency and quality of patient care, enabling faster decision-making and better-informed treatment plans. Additionally, this application is directed to preprocessing guideline documents and time series of user data files, automatically generating multiple customized prompts and results, and adaptively visualizing complex guideline policies and compliance conditions of the user data files. Thus, the computational resources of computers may be utilized efficiently in a practical application to provide accurate answers despite complexity of the guideline documents and the user data files.

These illustrative embodiments and implementations are mentioned not to limit or define the disclosure, but to provide examples to aid understanding thereof. Additional embodiments are discussed in the Detailed Description, and further description is provided there.

### BRIEF DESCRIPTION OF THE DRAWINGS

The features and advantages of the present invention will be more fully disclosed in, or rendered obvious by the following detailed description of the preferred embodiments, which are to be considered together with the accompanying drawings wherein like numbers refer to like parts.

FIG. 1 illustrates a network environment configured to provide a classification environment, in accordance with some embodiments.

FIG. 2 illustrates a block diagram of a computing device, in accordance with some embodiments.

FIG. 3 is another example of a data processing system for training and applying a neural network based (NN-based) data processing model for processing input data, in accordance with some embodiments.

FIG. 4A is an exemplary neural network (NN) applied to process input data in an NN-based data processing model, in accordance with some embodiments, and FIG. 4B is an example of a node in the NN, in accordance with some embodiments.

FIG. 5 is a structural diagram of a language model formed in a transformer architecture, in accordance with some embodiments.

FIG. 6 is a block diagram of an example computer system for reviewing user data files associated with a person with reference to a guideline document, in accordance with some embodiments.

FIG. 7 is a flow diagram of an example process of reviewing user data files associated with a person with reference to a guideline document, in accordance with some embodiments.

FIGS. 8A-8D is a flow diagram of an example process of processing user data files with reference to a guideline document, in accordance with some embodiments.

FIG. 9 illustrate excerpts of an example guideline document followed to generate a plurality of prompts for reviewing a braining imaging procedure, in accordance with some embodiments.

FIGS. 10A and 10B are example graphical user interfaces rendered to display an ordered list of guideline policies and a user data file during execution of a medical request assessment application, in accordance with some embodiments.

FIGS. 10C and 10D are example graphical user interfaces rendered to display a criterion that is satisfied by a user data file, in accordance with some embodiments.

FIG. 11 is a flow diagram of an example method for reviewing a time series of user data files with reference to a guideline document, in accordance with some embodiments.

FIG. 12 is a block diagram of an example network environment configured to manage patient health information, in accordance with some embodiments.

Like reference numerals refer to corresponding parts throughout the drawings.

### DETAILED DESCRIPTION

This description of the exemplary embodiments is intended to be read in connection with the accompanying drawings, which are to be considered part of the entire written description. Terms concerning data connections, coupling and the like refer to a relationship wherein systems or elements are logically connected to one another either directly or indirectly through intervening systems, unless expressly described otherwise. The term “operatively coupled” is such a coupling or connection that allows the pertinent structures to operate as intended by virtue of that relationship.

In the following, various embodiments are described with respect to the claimed systems as well as with respect to the claimed methods. Features, advantages, or alternative embodiments herein may be assigned to the other claimed objects and vice versa. In other words, claims for the systems may be improved with features described or claimed in the context of the methods. In this case, the functional features of the method are embodied by objective units of the systems. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

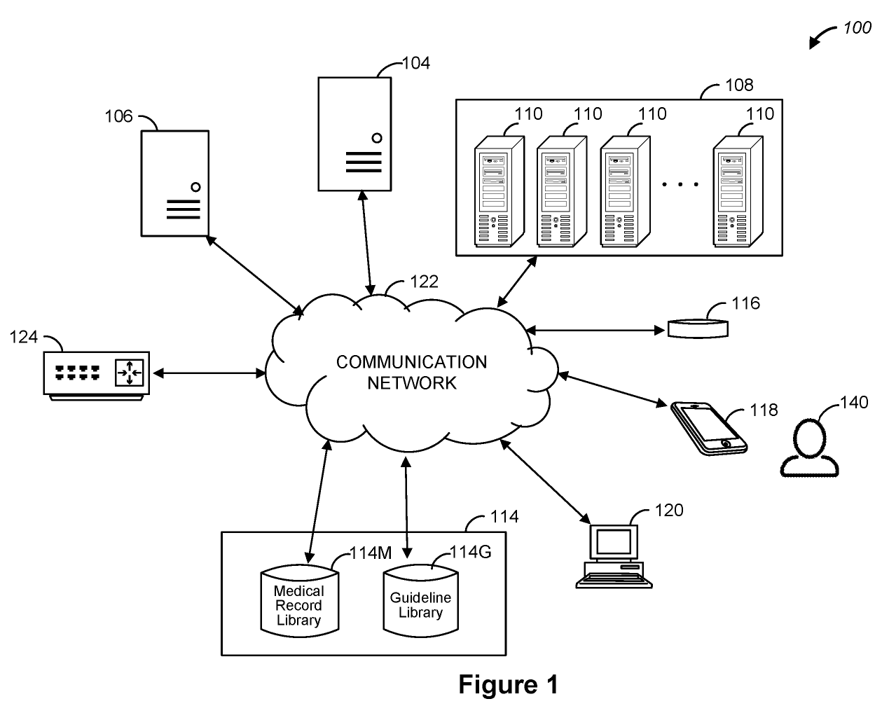

FIG. 1 illustrates a network environment 100 configured to provide a classification environment, in accordance with some embodiments. The network environment 100 includes a plurality of devices or systems configured to communicate over one or more network channels, illustrated as a network cloud 122. For example, in various embodiments, the network environment 100 may include, but is not limited to, a computing device 104, a web server 106, a cloud-based engine 108 including one or more processing devices 110, a database 114 (e.g., a medical record library 114M, a guideline library 114G), and/or one or more user computing devices 116, 118, 120 operatively coupled over the network 122. The computing device 104, the web server 106, the processing device(s) 110, and/or the user computing devices 116, 118, 120 may each be a suitable computing device that includes any hardware or hardware and software combination for processing and handling information. For example, each computing device may include, but is not limited to, one or more processors, one or more field-programmable gate arrays (FPGAs), one or more application-specific integrated circuits (ASICs), one or more state machines, digital circuitry, and/or any other suitable circuitry. In addition, each computing device may transmit and receive data over the communication network 122.

In some embodiments, each of the computing device 104 and the processing device(s) 110 may be a computer, a workstation, a laptop, a server such as a cloud-based server, or any other suitable device. In some embodiments, each of the processing devices 110 is a server that includes one or more processing units, such as one or more graphical processing units (GPUs), one or more central processing units (CPUs), and/or one or more processing cores. Each processing device 110 may, in some embodiments, execute one or more virtual machines. In some embodiments, processing resources (e.g., capabilities) of the one or more processing devices 110 are offered as a cloud-based service (e.g., cloud computing). For example, the cloud-based engine 108 may offer computing and storage resources of the one or more processing devices 110 to the computing device 104.

In some embodiments, each of the user computing devices 116, 118, 120 may be a cellular phone, a smart phone, a tablet, a personal assistant device, a voice assistant device, a digital assistant, a laptop, a computer, or any other suitable device. In some embodiments, the web server 106 hosts one or more network environments, such as an interactive chat network environment. In some embodiments, the computing device 104, the processing devices 110, and/or the web server 106 are operated by the network environment provider, and the user computing devices 116, 118, 120 are operated by users of the network environment. In some embodiments, the processing devices 110 are operated by a third party (e.g., a cloud-computing provider).

Although FIG. 1 illustrates three user computing devices 116, 118, 120, the network environment 100 may include any number of user computing devices 116, 118, 120. Similarly, the network environment 100 may include any number of the computing device 104, the web server 106, the processing devices 110, and/or the databases 114. It will further be appreciated that additional systems, servers, storage mechanism, etc. may be included within the network environment 100. In addition, although embodiments are illustrated herein having individual, discrete systems, it will be appreciated that, in some embodiments, one or more systems may be combined into a single logical and/or physical system. For example, in various embodiments, one or more of the computing devices 104, the web server 106, the database 114, the user computing devices 116, 118, 120, and/or the router 124 may be combined into a single logical and/or physical system. Similarly, although embodiments are illustrated having a single instance of each device or system, it will be appreciated that additional instances of a device may be implemented within the network environment 100. In some embodiments, two or more systems may be operated on shared hardware in which each system operates as a separate, discrete system utilizing the shared hardware, for example, according to one or more virtualization schemes.

The communication network 122 may be a WiFi® network, a cellular network such as a 3GPP network, a Bluetooth network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. The communication network 122 may provide access to, for example, the Internet.

Each of the user computing devices 116, 118, 120 may communicate with the web server 106 over the communication network 122. For example, each of the user computing devices 116, 118, 120 may be operable to view, access, and interact with a website, such as an interactive chat website, hosted by the web server 106. The web server 106 may transmit user session data related to a user’s activity (e.g., interactions) on the website. For example, a user may operate one of the user computing devices 116, 118, 120 to initiate a web browser that is directed to the website hosted by the web server 106. The website may also allow the user to interact with one or more of interface elements to perform specific operations, such as providing one or more responses to a query.

In some embodiments, the computing device 104 may execute one or more models, processes, or algorithms, such as a machine learning model, deep learning model, statistical model, generative model, etc., for content extraction, summarization, classification, or similar tasks. The computing device 104 is further operable to communicate with the database(s) 114 over the communication network 122. For example, the computing device 104 may store data to, and read data from, the database 114. The database 114 may be a remote storage device, such as a cloud-based server, a disk (e.g., a hard disk), a memory device on another application server, a networked computer, or any other suitable remote storage. Although shown remote to the computing device 104, in some embodiments, the database 114 may be a local storage device, such as a hard drive, a non-volatile memory, or a USB stick. The computing device 104 may store interaction data received from the web server 106 in the database 114. The computing device 104 may also receive from the web server 106 user session data identifying events associated with user interactions, and may store the user session data in the database 114.

The computing device 104 and/or one or more of the processing devices 110 may train one or more models based on corresponding training data. The computing device 104 may store the models in a database, such as in the database 114 (e.g., a cloud storage database). The models, when executed by the computing device 104, allow the computing device 104 to perform one or more operations, such as a summary generation operation with a user via a chat interface. For example, the computing device 104 may obtain one or more models from the database 114.

In some embodiments, the computing device 104 assigns the models (or parts thereof) for execution to one or more processing devices 110. For example, each model may be assigned to a virtual machine hosted by a processing device 110. The virtual machine may cause the models or parts thereof to execute on one or more processing units such as GPUs. In some embodiments, the virtual machines assign each model (or part thereof) among a plurality of processing units.

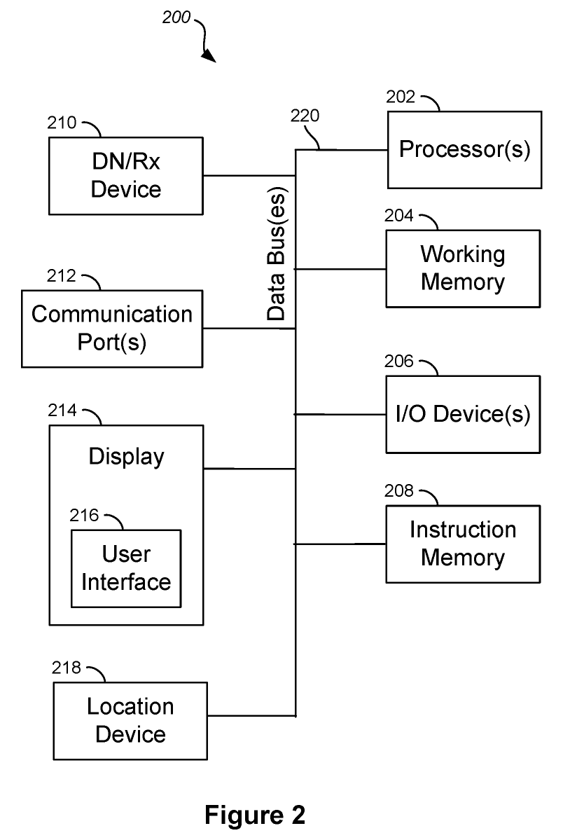

FIG. 2 illustrates a block diagram of a computing device 200, in accordance with some embodiments. In some embodiments, each of the computing device 104, the web server 106, the one or more processing devices 110, and/or the user computing devices 116, 118, 120 in FIG. 2 may include the features shown in FIG. 2. Although FIG. 2 is described with respect to certain components shown therein, it will be appreciated that the elements of the computing device 200 may be combined, omitted, and/or replicated. In addition, it will be appreciated that additional elements other than those illustrated in FIG. 2 may be added to the computing device.

As shown in FIG. 2, the computing device 200 may include one or more processors 202, an instruction memory 204, a working memory 206, one or more input/output devices 208, a transceiver 210, one or more communication ports 212, a display 214 with a user interface 216, and an optional location device 218, all operatively coupled to one or more data buses 220. The data buses 220 allow for communication among the various components. The data buses 220 may include wired, or wireless, communication channels.

The one or more processors 202 may include any processing circuitry operable to control operations of the computing device 200. In some embodiments, the one or more processors 202 include one or more distinct processors, each having one or more cores (e.g., processing circuits). Each of the distinct processors may have the same or different structure. The one or more processors 202 may include one or more central processing units (CPUs), one or more graphics processing units (GPUs), application specific integrated circuits (ASICs), digital signal processors (DSPs), a chip multiprocessor (CMP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, and/or a very long instruction word (VLIW) microprocessor, or other processing device. The one or more processors 202 may also be implemented by a controller, a microcontroller, an application specific integrated circuit (ASIC), a field programmable gate array (FPGA), a programmable logic device (PLD), etc.

In some embodiments, the one or more processors 202 are configured to implement an operating system (OS) and/or various applications. Examples of an OS include, for example, operating systems generally known under various trade names such as Apple macOS™, Microsoft Windows™, Android™, Linux™, and/or any other proprietary or open-source OS. Examples of applications include, for example, network applications, local applications, data input/output applications, user interaction applications, etc.

The instruction memory 204 may store instructions that are accessed (e.g., read) and executed by at least one of the one or more processors 202. For example, the instruction memory 204 may be a non-transitory, computer-readable storage medium such as a read-only memory (ROM), an electrically erasable programmable read-only memory (EEPROM), flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. The one or more processors 202 may be configured to perform a certain function or operation by executing code, stored on the instruction memory 204, embodying the function or operation. For example, the one or more processors 202 may be configured to execute code stored in the instruction memory 204 to perform one or more of any function, method, or operation disclosed herein.

Additionally, the one or more processors 202 may store data to, and read data from, the working memory 206. For example, the one or more processors 202 may store a working set of instructions to the working memory 206, such as instructions loaded from the instruction memory 204. The one or more processors 202 may also use the working memory 206 to store dynamic data created during one or more operations. The working memory 206 may include, for example, random access memory (RAM) such as a static random access memory (SRAM) or dynamic random access memory (DRAM), Double-Data-Rate DRAM (DDR-RAM), synchronous DRAM (SDRAM), an EEPROM, flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. Although embodiments are illustrated herein including separate instruction memory 204 and working memory 206, it will be appreciated that the computing device 200 may include a single memory unit configured to operate as both instruction memory and working memory. Further, although embodiments are discussed herein including non-volatile memory, it will be appreciated that computing device 200 may include volatile memory components in addition to at least one non-volatile memory component.

In some embodiments, the instruction memory 204 and/or the working memory 206 includes an instruction set, in the form of a file for executing various methods, such as methods for summary generation interactions, as described herein. The instruction set may be stored in any acceptable form of machine-readable instructions, including source code or various appropriate programming languages. Some examples of programming languages that may be used to store the instruction set include, but are not limited to: Java, JavaScript, C, C++, C#, Python, Objective-C, Visual Basic, .NET, HTML, CSS, SQL, NoSQL, Rust, Perl, etc. In some embodiments a compiler or interpreter is configured to convert the instruction set into machine executable code for execution by the one or more processors 202.

The input-output devices 208 may include any suitable device that allows for data input or output. For example, the input-output devices 208 may include one or more of a keyboard, a touchpad, a mouse, a stylus, a touchscreen, a physical button, a speaker, a microphone, a keypad, a click wheel, a motion sensor, a camera, and/or any other suitable input or output device.

The transceiver 210 and/or the communication port(s) 212 allow for communication with a network, such as the communication network 122 of FIG. 1. For example, if the communication network 122 of FIG. 1 is a cellular network, the transceiver 210 is configured to allow communications with the cellular network. In some embodiments, the transceiver 210 is selected based on the type of the communication network 122 the computing device 200 will be operating in. The one or more processors 202 are operable to receive data from, or send data to, a network, such as the communication network 122 of FIG. 1, via the transceiver 210.

The communication port(s) 212 may include any suitable hardware, software, and/or combination of hardware and software that is capable of coupling the computing device 200 to one or more networks and/or additional devices. The communication port(s) 212 may be arranged to operate with any suitable technique for controlling information signals using a desired set of communications protocols, services, or operating procedures. The communication port(s) 212 may include the appropriate physical connectors to connect with a corresponding communications medium, whether wired or wireless, for example, a serial port such as a universal asynchronous receiver/transmitter (UART) connection, a Universal Serial Bus (USB) connection, or any other suitable communication port or connection. In some embodiments, the communication port(s) 212 allows for the programming of executable instructions in the instruction memory 204. In some embodiments, the communication port(s) 212 allow for the transfer (e.g., uploading or downloading) of data, such as machine learning model training data.

In some embodiments, the communication port(s) 212 are configured to couple the computing device 200 to a network. The network may include local area networks (LAN) as well as wide area networks (WAN) including without limitation Internet, wired channels, wireless channels, communication devices including telephones, computers, wire, radio, optical and/or other electromagnetic channels, and combinations thereof, including other devices and/or components capable of/associated with communicating data. For example, the communication environments may include in-body communications, various devices, and various modes of communications such as wireless communications, wired communications, and combinations of the same.

In some embodiments, the transceiver 210 and/or the communication port(s) 212 are configured to utilize one or more communication protocols. Examples of wired protocols may include, but are not limited to, Universal Serial Bus (USB) communication, RS-232, RS-422, RS-423, RS-485 serial protocols, FireWire, Ethernet, Fibre Channel, MIDI, ATA, Serial ATA, PCI Express, T-1 (and variants), Industry Standard Architecture (ISA) parallel communication, Small Computer System Interface (SCSI) communication, or Peripheral Component Interconnect (PCI) communication, etc. Examples of wireless protocols may include, but are not limited to, the Institute of Electrical and Electronics Engineers (IEEE) 802.xx series of protocols, such as IEEE 802.11a/b/g/n/ac/ag/ax/be, IEEE 802.16, IEEE 802.20, GSM cellular radiotelephone system protocols with GPRS, CDMA cellular radiotelephone communication systems with 1xRTT, EDGE systems, EV-DO systems, EV-DV systems, HSDPA systems, Wi-Fi Legacy, Wi-Fi 1/2/3/4/5/6/6E, wireless personal area network (PAN) protocols, Bluetooth Specification versions 5.0, 6, 7, legacy Bluetooth protocols, passive or active radio-frequency identification (RFID) protocols, Ultra-Wide Band (UWB), Digital Office (DO), Digital Home, Trusted Platform Module (TPM), ZigBee, etc.

The display 214 may be any suitable display, and may display the user interface 216. The user interfaces 216 may enable user interaction with a first generative model. For example, the user interface 216 may be a user interface for an application of a network environment operator that allows a user to view and interact with the operator’s website. In some embodiments, a user may interact with the user interface 216 by engaging the input-output devices 208. In some embodiments, the display 214 may be a touchscreen, where the user interface 216 is displayed on the touchscreen.

The display 214 may include a screen such as, for example, a Liquid Crystal Display (LCD) screen, a light-emitting diode (LED) screen, an organic LED (OLED) screen, a movable display, a projection, etc. In some embodiments, the display 214 may include a coder/decoder, also known as Codecs, to convert digital media data into analog signals. For example, the visual peripheral output device may include video Codecs, audio Codecs, or any other suitable type of Codec.

The optional location device 218 may be communicatively coupled to a location network and operable to receive position data from the location network. For example, in some embodiments, the location device 218 includes a GPS device configured to receive position data identifying a latitude and longitude from one or more satellites of a GPS constellation. As another example, in some embodiments, the location device 218 is a cellular device configured to receive location data from one or more localized cellular towers. Based on the position data, the computing device 200 may determine a local geographical area (e.g., town, city, state, etc.) of its position.

In some embodiments, the computing device 200 is configured to implement one or more modules or engines, each of which is constructed, programmed, configured, or otherwise adapted, to autonomously conduct a function or set of functions. A module/engine may include a component or arrangement of components implemented using hardware, such as by an application specific integrated circuit (ASIC) or field-programmable gate array (FPGA), for example, or as a combination of hardware and software, such as by a microprocessor system and a set of program instructions that adapt the module/engine to implement the particular functionality, which (while being executed) transform the microprocessor system into a special-purpose device. A module/engine may also be implemented as a combination of the two, with certain functions facilitated by hardware alone, and other functions facilitated by a combination of hardware and software. In certain implementations, at least a portion, and in some cases, all, of a module/engine may be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each module/engine may be realized in a variety of physically realizable configurations, and should generally not be limited to any particular implementation exemplified herein, unless such limitations are expressly called out. In addition, a module/engine may itself be composed of more than one sub-modules or sub-engines, each of which may be regarded as a module/engine in its own right. Moreover, in the embodiments described herein, each of the various modules/engines corresponds to a defined autonomous functionality; however, it should be understood that in other contemplated embodiments, each functionality may be distributed to more than one module/engine. Likewise, in other contemplated embodiments, multiple defined functionalities may be implemented by a single module/engine that performs those multiple functions, possibly alongside other functions, or distributed differently among a set of modules/engines than specifically illustrated in the embodiments herein.

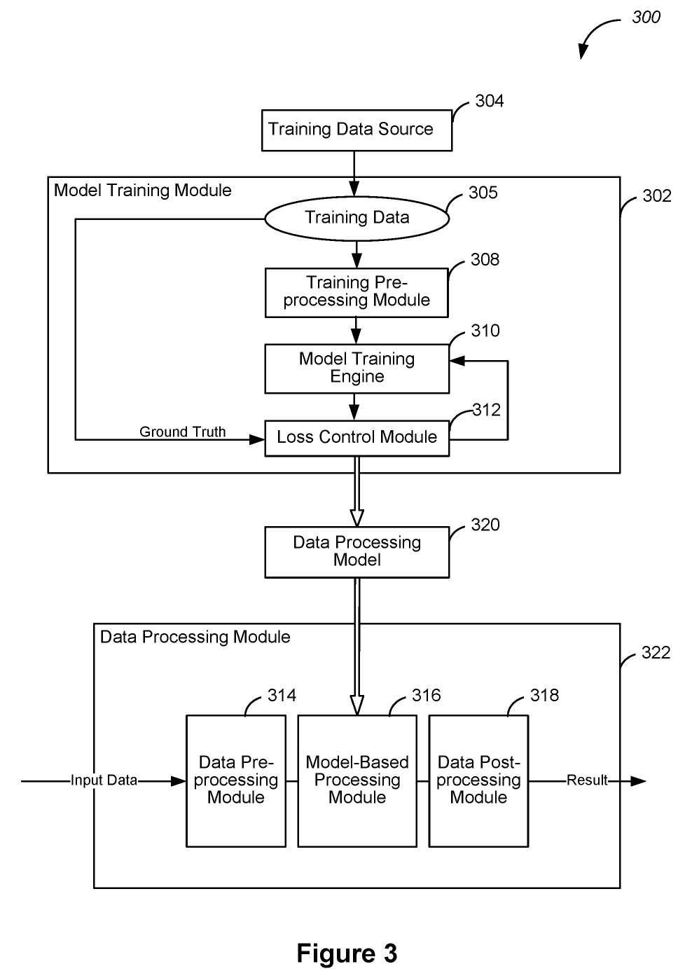

FIG. 3 is another example of a data processing system 300 for training and applying an NN-based data processing model 320 for processing input data, in accordance with some embodiments. The data processing system 300 includes a model training module 302 for establishing the data processing model 320 and a data processing module 322 for processing the input data using the data processing model 320. In some embodiments, both of the model training module 302 and the data processing module 322 are located on a client device 104 of the data processing system 300, while a training data source 304 distinct from the client device 104 provides training data 305 to the client device 104. The training data source 304 is optionally a server or storage. Alternatively, in some embodiments, the model training module 302 and the data processing module 322 are both located on a server of the data processing system 300. The training data source 304 providing the training data 305 is optionally the server itself, another server, or the storage 106. Additionally, in some embodiments, the model training module 302 and the data processing module 322 are separately located on a server and client device 104, and the server provides the trained data processing model 320 to the client device 104.

The model training module 302 includes one or more training pre-processing modules 308, a model training engine 310, and a loss control module 312. The data processing model 320 is trained according to the type of input data to be processed. The training data 305 is consistent with the type of the input data, so is a training pre-processing module 308 applied to process the training data 305, e.g., converting the training data 305 to a uniform data format or data structure. The model training engine 310 receives pre-processed training data 305 provided by the training pre-processing modules 308, further processes the pre-processed training data 305 using an existing data processing model 320, and generates an output from each training data item. During this course, the loss control module 312 can monitor a loss function comparing the output associated with the respective training data item and a ground truth of the respective training data item. The model training engine 310 modifies the data processing model 320 to reduce the loss function, until the loss function satisfies a loss criterion (e.g., a comparison result of the loss function is minimized or reduced below a loss threshold). The modified data processing model 320 is provided to the data processing module 322 to process the input data.

In some embodiments, the model training module 302 offers supervised learning in which the training data 305 is entirely labelled and includes a desired output for each training data item (also called the ground truth in some situations). Conversely, in some embodiments, the model training module 302 offers unsupervised learning in which the training data 305 are not labelled. The model training module 302 is configured to identify previously undetected patterns in the training data 305 without pre-existing labels and with no or little human supervision. Additionally, in some embodiments, the model training module 302 offers partially supervised learning in which the training data 305 are partially labelled.

The data processing module 322 includes a data pre-processing module 314, a model-based processing module 316, and a data post-processing module 318. The data pre-processing modules 314 pre-processes the input data based on the type of the input data. Functions of the data pre-processing modules 314 are consistent with those of the pre-processing modules 308 and covert the input data to a predefined content format that is acceptable by inputs of the model-based processing module 316. The model-based processing module 316 applies the trained data processing model 320 provided by the model training module 302 to process the pre-processed input data. The model-based processing module 316 can also monitor an error indicator to determine whether the input data has been properly processed in the data processing module 322. In some embodiments, the processed input data is further processed by the data post-processing module 318 to present the processed input data in a preferred format or to provide other related information that can be derived from the processed input data.

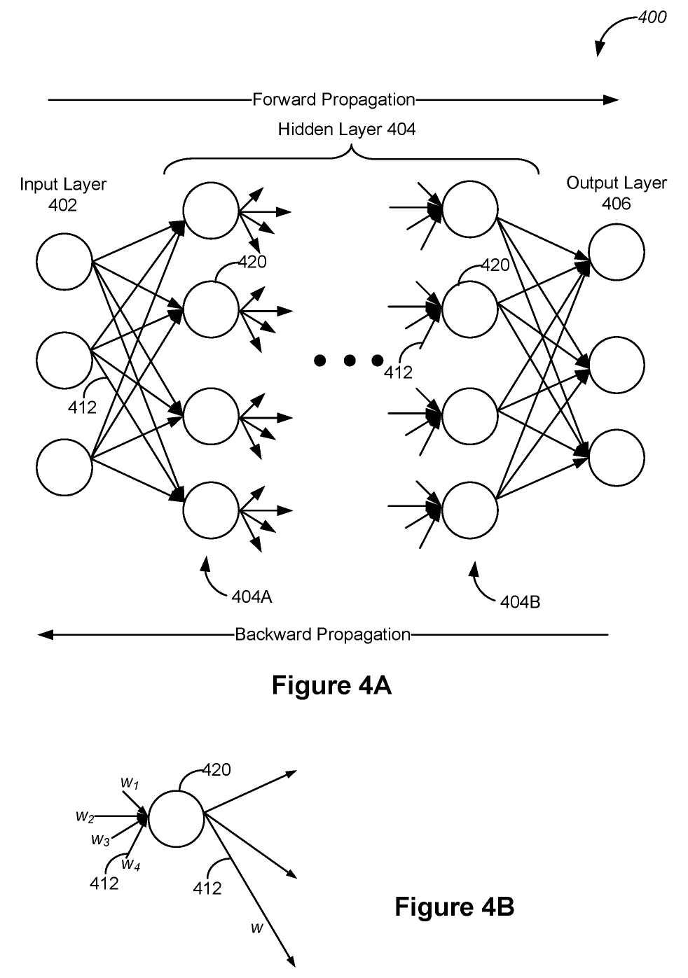

FIG. 4A is an exemplary neural network (NN) 400 applied to process input data in an NN-based data processing model 320, in accordance with some embodiments, and FIG. 4B is an example of a node 420 in the NN 400, in accordance with some embodiments. The data processing model 320 is established based on the neural network 400. A corresponding model-based processing module 316 applies the data processing model 320 including the neural network 400 to process input data that has been converted to a predefined content format. The neural network 400 includes a collection of nodes 420 that are connected by links 412. Each node 420 receives one or more node inputs and applies a propagation function to generate a node output from the node input(s). As the node output is provided via one or more links 412 to one or more other nodes 420, a weight w associated with each link 412 is applied to the node output. Likewise, the node input(s) can be combined based on corresponding weights w 1, w 2, w 3, and w 4 according to the propagation function. For example, the propagation function is a product of a non-linear activation function and a linear weighted combination of the node input(s).

The collection of nodes 420 is organized into one or more layers in the neural network 400. Optionally, the layer(s) may include a single layer acting as both an input layer and an output layer. Optionally, the layer(s) may include an input layer 402 for receiving inputs, an output layer 406 for providing outputs, and zero or more hidden layers 404 (e.g., 404A and 404B) between the input and output layers 402 and 406. A deep neural network has more than one hidden layer 404 between the input and output layers 402 and 406. In the neural network 400, each layer is only connected with its immediately preceding and/or immediately following layer. In some embodiments, a layer 402 or 404B is a fully connected layer because each node 420 in the layer 402 or 404B is connected to every node 420 in its immediately following layer. In some embodiments, one of the hidden layers 404 includes two or more nodes that are connected to the same node in its immediately following layer for down sampling or pooling the nodes 420 between these two layers. Particularly, max pooling uses a maximum value of the two or more nodes in the layer 404B for generating the node of the immediately following layer 406 connected to the two or more nodes.

In some embodiments, a convolutional neural network (CNN) is applied in a data processing model 320 to process input data. The CNN employs convolution operations and belongs to a class of deep neural networks 400, i.e., a feedforward neural network that only moves data forward from the input layer 402 through the hidden layers to the output layer 406. The hidden layer(s) of the CNN can be convolutional layers convolving with multiplication or dot product. Each node in a convolutional layer receives inputs from a receptive area associated with a previous layer (e.g., five nodes), and the receptive area is smaller than the entire previous layer and may vary based on a location of the convolution layer in the convolutional neural network.

Alternatively and additionally, in some embodiments, a recurrent neural network (RNN) is applied in the data processing model 320 to process input data (particularly, textual and speech data). Nodes in successive layers of the RNN follow a temporal sequence, such that the RNN exhibits a temporal dynamic behavior. For example, each node 420 of the RNN has a time-varying real-valued activation. Examples of the RNN include, but are not limited to, a long short-term memory (LSTM) network, a fully recurrent network, an Elman network, a Jordan network, a Hopfield network, a bidirectional associative memory (BAM network), an echo state network, an independently RNN (IndRNN), a recursive neural network, and a neural history compressor. In some embodiments, the RNN can be used for handwriting or speech recognition. It is noted that in some embodiments, two or more types of input data are processed by the data processing module 322, and two or more types of neural networks (e.g., both CNN and RNN) are applied to process the input data jointly.

The training process is a process for calibrating all of the weights w i for each layer of the data processing model 320 using a training data set which is provided in the input layer 402. The training process typically includes two steps, forward propagation and backward propagation, which are repeated multiple times until a predefined convergence condition is satisfied. In the forward propagation, the set of weights for different layers are applied to the input data and intermediate results from the previous layers. In the backward propagation, a margin of error of the output (e.g., a loss function) is measured, and the weights are adjusted accordingly to decrease the error. The activation function is optionally linear, rectified linear unit, sigmoid, hyperbolic tangent, or of other types. In some embodiments, a network bias term b is added to the sum of the weighted outputs from the previous layer before the activation function is applied. The network bias b provides a perturbation that helps the NN 400 avoid over fitting the training data. The result of the training includes the network bias parameter b for each layer.

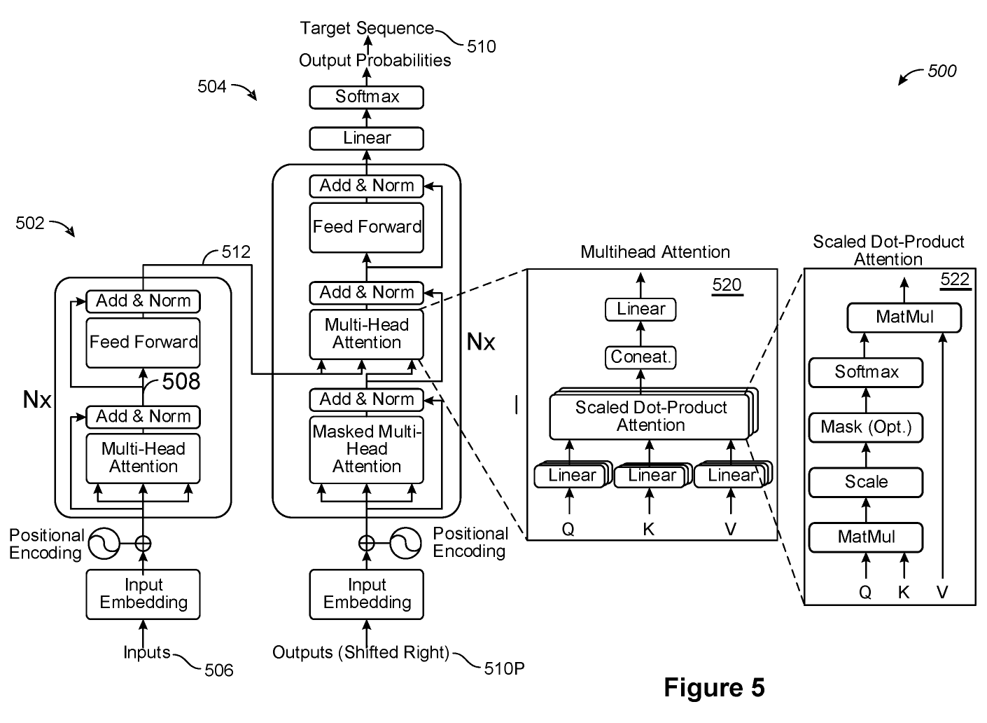

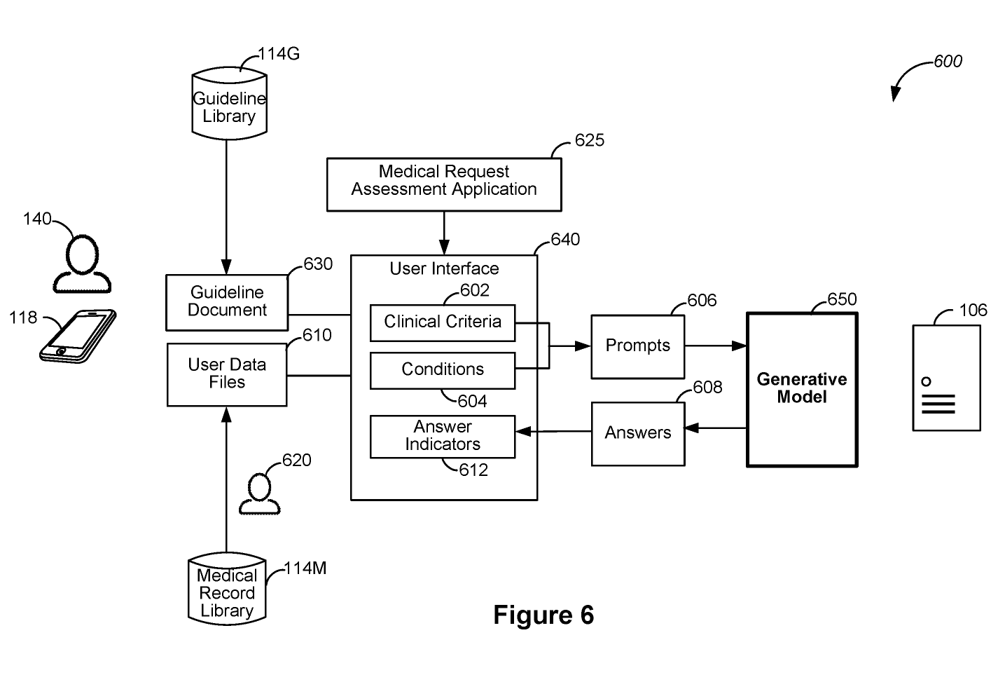

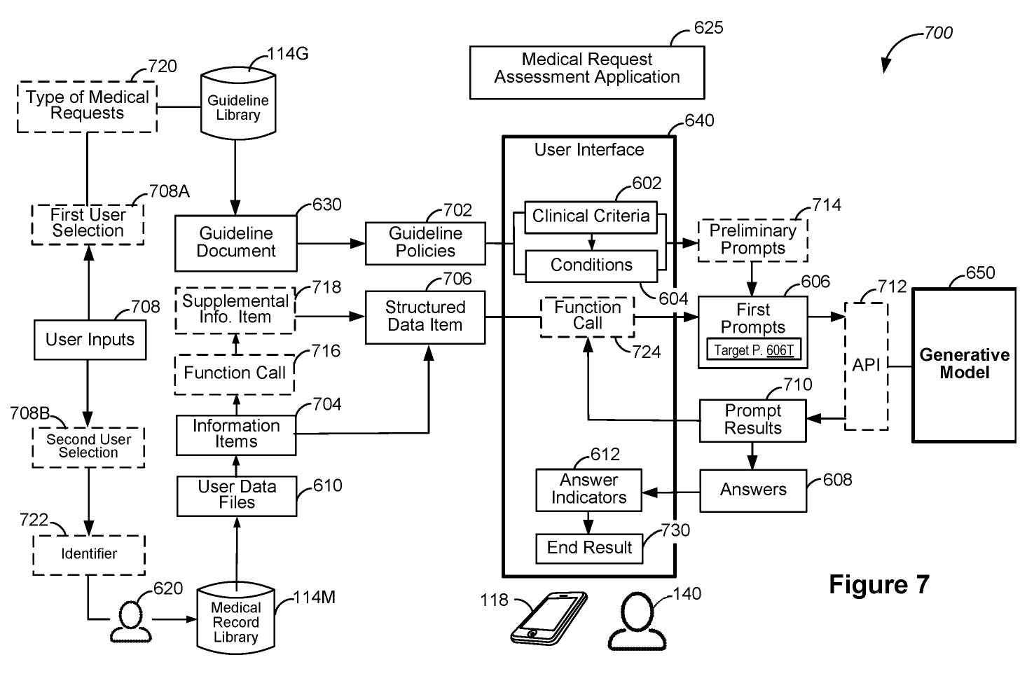

FIG. 5 is a structural diagram of a language model 500 formed in a transformer architecture, in accordance with some embodiments. Generative AI uses natural language processing (NLP) and machine learning to create natural language data or content. The language model 500 includes a deep learning neural network configured to perform natural language processing (NLP) tasks, e.g., text generation, summarization, translation, text classification, and answering questions, thereby enabling Generative AI. In some embodiments, the language model 500 includes an LLM. Compared with a normal language model, the LLM includes more than 100 million parameters, and is pre-trained with large corpora of text. In some embodiments, the language model 500 is implemented with a transformer architecture, and configured to shift through large datasets and recognize patterns and relationships between words or phrases. The transformer architecture includes an attention mechanism that weighs the importance of different words or phrases in a given context. In some embodiments, a generative model 650 (FIGS. 6 and 7) is implemented as a language model 500.

The transformer architecture of the language model 500 includes an encoder network 502 and a decoder network 504. The encoder network 502 is configured to receive an input sequence 506 and generate a sequence of hidden states 508. Each hidden state 508 includes a vector that encodes contextual information of a word in the input sequence 506 based on its relative position. The decoder network 504 is configured to receive portions 510P of a target sequence 510 successively and use an output 512 of the encoder network 502 to generate the target sequence 510. In some embodiments, the decoder network 504 starts with a starting token (e.g., “start”) and generates one prediction at a time. The decoder network 504 uses the output 512 produced by the encoder network 502 to understand the context of the input sequence 506. For each word of the target sequence 510 to be predicted, the decoder network 504 uses cross-attention mechanisms to focus on corresponding portions of the output 512 of the encoder network 502. As each word of the target sequence 510 is generated, the decoder network 504 updates its state and predicts a next word, until the entire target sequence 510 is generated.

In some embodiments, the language model 500 applies a self-attention mechanism, and each position in a sequence (e.g., a natural language query) is attended to all positions in the same sequence. Self-attention helps the language model 500 to understand and interpret the sequence by considering the entire sequence. For instance, when processing the natural language query, self-attention allows each word to be contextualized in relation to every other word in that natural language query. Alternatively, in some embodiments, the language model 500 applies a transformer architecture including multihead attention 520 (also called multihead self-attention). Each attention head 522 learns a respective attention mechanism so that multihead attention 520 as a whole can learn more complex relationships. For example, referring to FIG. 5, multihead attention 520 is applied in both the encoder network 502 and the decoder network 504 of the language model 500 implemented in the transformer architecture.

FIG. 6 is a block diagram of an example computer system 600 for reviewing user data files 610 associated with a person 620 with reference to a guideline document 630, in accordance with some embodiments. The computer system 600 may include one or more of: a computing device 104, a web server 106, a cloud-based engine 108 including one or more processing devices 110, a database 114 (e.g., a medical record library 114M, a guideline library 114G), and one or more user computing devices 116, 118, 120 operatively coupled over a network 122. A medical request assessment application 625 is executed by the computer system 600, and renders a user interface 640 for receiving user inputs from a user 140 and presenting information to the user 140. The guideline library 114G stores a plurality of guideline documents 630 that are created independently of any specific patients. One or more clinical criteria 602 and a plurality of conditions 604 associated with each clinical criterion 602 are extracted from a guideline document 630, and displayed on the user interface 640. In some embodiments, the medical record library 114M stores a time series of user data files 610 associated the person 620 (e.g., a temporal sequence of medical records regarding the person’s visits to a doctor office).

In some embodiments, user inputs are entered on the user interface 640 (e.g., via a user computing device 116, 118, and 120) to select the user data files 610 of the person 620 and the guideline document 630. Automatically and without user intervention, the computer system 600 generates a plurality of first prompts 606 associated with the person 620 based on the one or more clinical criteria 602 and the plurality of conditions 604 associated with each clinical criterion 602. A generative model 650 is applied to generate prompt results leading to a plurality of answers 608 in response to the plurality of first prompts 606 based on the user data files 610 associated with the person 620. More specifically, in some embodiments, the generative model 650 is used to review medical records of the person 620 to determine whether the medical records of the person 620 meet with the plurality of conditions 604 of at least one of the one or more criteria 602. The user interface 640 is modified based on the plurality of answers 608. For example, the one or more clinical criteria 602 and a plurality of conditions 604 associated with each clinical criterion 602 are displayed on an ordered sequence of guideline policies, and a plurality of answer indicators 612 are displayed on the user interface 640.

FIG. 7 is a flow diagram of an example process 700 of reviewing user data files 610 associated with a person 620 with reference to a guideline document 630, in accordance with some embodiments. The process 700 is implemented at a computer system 600 having one or more processors and memory for storing one or more instructions. The computer system 600 obtains a guideline document 630 for assessing a type 720 of medical requests (e.g., brain Computed Tomography (CT) imaging, genetic testing during pregnancy), and generates an ordered list of guideline policies 702 based on the guideline document 630, e.g., independently of information of any particular patients. The ordered list of guideline policies 702 includes one or more criteria 602 and a plurality of conditions 604 associated with each criterion 602. Each guideline policy 702 corresponds to at least a respective condition 604 of a respective criterion 602.

The computer system 600 also obtains time series of user data files 610 associated with a plurality of patients (e.g., a person 620). Each user data file 610 includes unstructured data of a respective person 620. For example, a user data file 610 is generated for each doctor visit of the person 620, and a time series of user data files 610 may be generated successively by different medical practitioners’ computers and stored in a medical record library 114M. In some embodiments, the time series of user data files 610 associated with a particular person 620 are provided to the computer system 600 jointly, e.g., in response to a data update request, at a time of submitting a medical assessment request. Alternatively, in some embodiments, each user data file 610 of the particular person 620 is separately submitted by a respective medical practitioner’s computer and stored in the medical record library 114M when the user data file 610 is available. The computer system 600 retrieves a plurality of information items 704 from the unstructured data of each user data file 610 to generate a structured data item 706 associated with the time series of user data files 610, e.g., independently of which guideline document is applied.

The computer system 600 executes a medical request assessment application 625 and renders a graphical user interface 640 via the assessment application 625. A guideline document 630, user data files 610, guideline policies 702 (e.g., criteria 602 and conditions 604), and assessment answers 608 are presented in a user-friendly manner on the user interface 640. A generative model 650 is applied to process a plurality of prompts 606 and generate prompt results resulting in a plurality of answers 608, e.g., according to a prompt graph, while the prompts 606 and the prompt results may be hidden from a user 140 of the medical request assessment application 625. More specifically, the computer system 600 receives one or more user inputs 708 of information identifying the guideline document 630 and the user data files 610 on the user interface 640. For example, the user input(s) 708 may select the guideline document 630 from a plurality of guideline documents that are available in a guideline library 114G. In another example, the user input(s) 708 may select the particular person 620 from a plurality of patients whose user data files are stored in the medical record library 114M. In response to the one or more user inputs 708, the ordered list of guideline policies 702 associated with the guideline document 630 are displayed on the user interface 640. Automatically and without user intervention, the computer system 600 generates a plurality of first prompts 606 based on the ordered list of guideline policies 702 and the structured data item 706 of the user data files 610. Each guideline policy 702 corresponds to a set of one or more respective first prompts 606. That said, in some embodiments, one of the plurality of guideline policies 702 may be associated with more than one prompt 606. In some embodiments, one of the plurality of first prompts 606 may be associated with more than one conditions 604 or more than criterion 602.

The computer system 600 further applies a generative model 650 (e.g., a transformer-based large language model 500 in FIG. 5) to generate a plurality of prompt results 710 in response to the plurality of first prompts 606 based on the user data files 610 and the structure data item 706. In some embodiments, the generative model 650 is executed by a server 106 that is associated with the medical request assessment application 625. Alternatively, in some embodiments, the generative model 650 is executed by a third-party server 106, independently of the medical request assessment application 625. The medical request assessment application 625 includes an application programming interface (API) 712 for applying the generative model 650 executed by the third-party server 106. Examples of the generative model 650 includes, but are not limited to, ChatGPT, Vertex AI, Gemini, and LLaMA. A plurality of answers 608 are determined in response to the ordered list of guideline policies 702 based on the prompt results 710. Each answer 608 corresponds to a respective guideline policy 702, and is determined based on one or more prompt results 710 of the set of one or more respective first prompts 606. Based on the answers 608, display of the ordered list of guideline policies 702 is updated, e.g., to incorporate associated answer indicators 612 on the user interface 640.

In some embodiments, the guideline document 630 is obtained and the ordered list of guideline policies 702 is generated in response to the one or more user inputs 708. Alternatively, in some embodiments, the ordered list of guideline policies 702 is generated and stored in the guideline library 114G, before the one or more user inputs 708 are received. The ordered list of guideline policies 702 is extracted from the guideline library 114G in response to the one or more user inputs 708. Further, in some embodiments, the guideline library 114G stores a plurality of preliminary prompts 714 associated with the ordered list of guideline policies 702. The preliminary prompts 714 are generated based on the guideline document 630, independently of the one or more user inputs 708. In response to the one or more user inputs 708, the computer system 600 extracts the plurality of preliminary prompts 714 from the guideline library 114G and modifies the plurality of preliminary prompts 714 based on the structured data item 706 of the user data files 610 to generate the plurality of first prompts 606. Stated another way, the guideline document 630 may be stored with preliminary prompts 714 that are prepared independently of the person 620, and the preliminary prompts 714 may be customized for the person 620 during the course of assessing the user data files 610 of the person 620.

In some embodiments, after obtaining the user data files 610 of the person 620, the computer system 600 applies a function call 716 to process a subset of the information items 704 and generate a supplemental information item 718, and consolidates the plurality of information items 704 and the supplemental information item 718 to generate the structured data item 706 associated with the user data files 610. For example, the function call 716 may be associated with a user application managing access to a public data source to obtain medical research results regarding similar patients to the person 620.

In some embodiments, the medical record library 114M stores the user data files 610 and the structured data item 706 associated with the person 620. The user data files 610 and the structured data item 706 associated with the person 620 are extracted from the medical record library 114M in response to the one or more user inputs 708.

In some embodiments, the one or more user inputs 708 include a first user selection 708A of the type 720 of medical requests. In response to the first user selection 708A, the computer system 600 identifies the guideline document 630 for assessing the type 720 of medical requests; and extracts the ordered list of guideline policies 702 associated with the guideline document 630. Alternatively or additionally, in some embodiments, the one or more user inputs 708 include receiving a second user selection 708B of an identifier 722 of the person 620. In response to the second user selection 708B, the computer system 600 extracts the user data files 610 and the structured data item 706 of the person 620 from the medical record library 114M. The plurality of first prompts 606 are generated based on the user data files 610 and the structured data item 706, and the generative model 650 is applied to process the first prompts 606 to generate the plurality of prompt results 710. Additionally, in some embodiments, the plurality of first prompts 606 include a target prompt associated with one of the plurality of conditions 604 of the one or more criteria 602. A function call 724 is applied to at least the structured data item 706 to generate a function result, and the target prompt 606T is generated based on the function result.

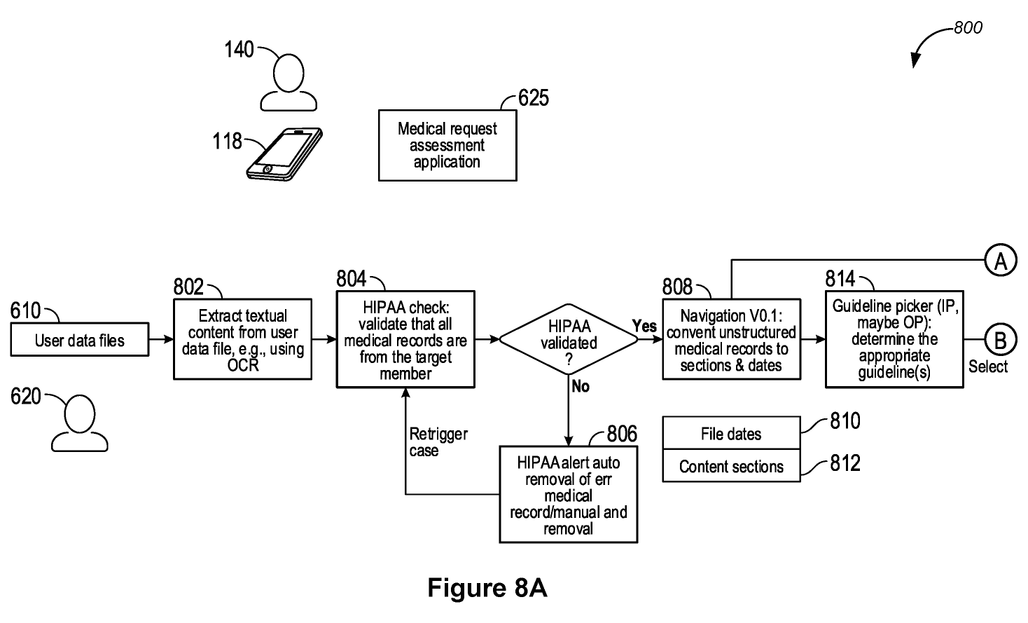

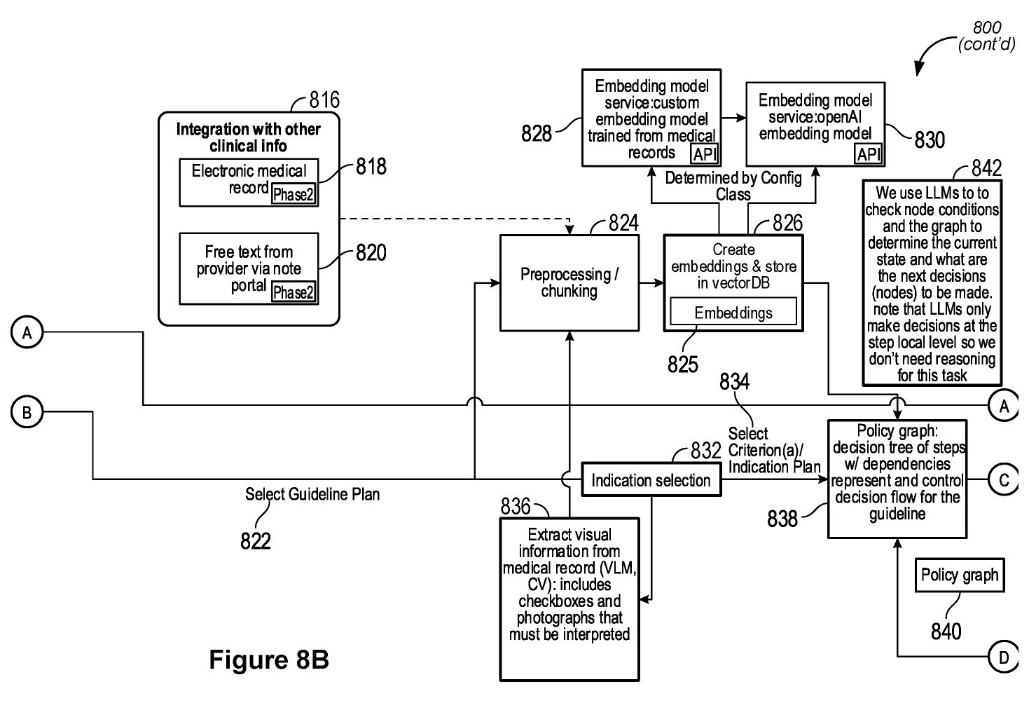

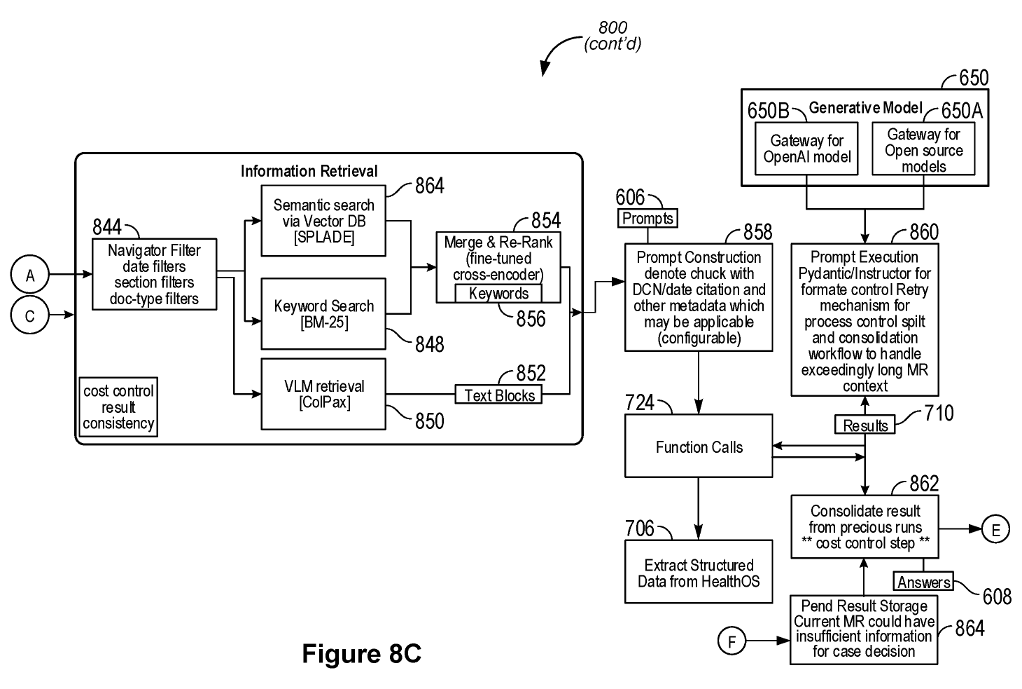

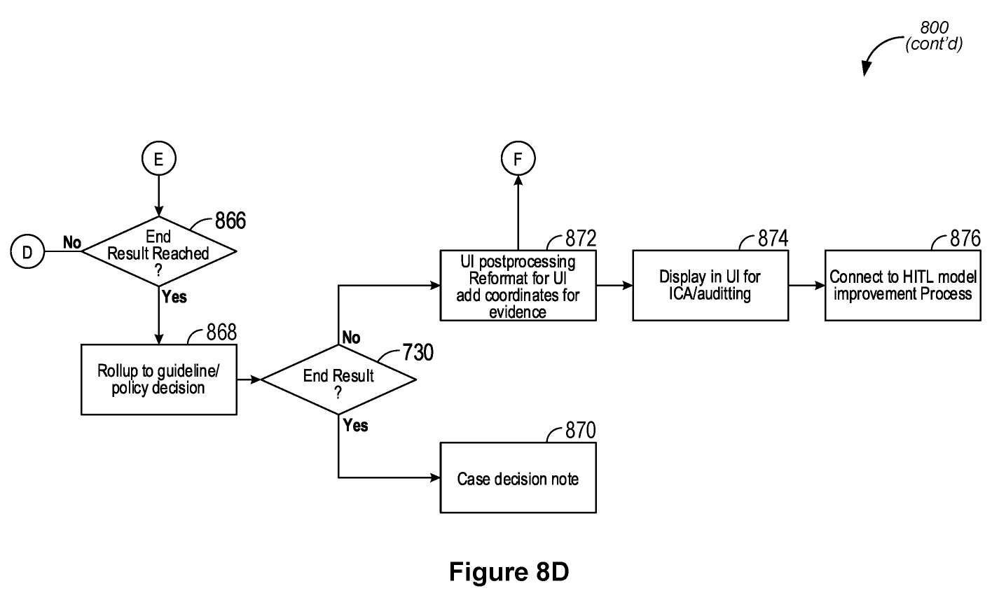

FIGS. 8A-8D is a flow diagram of an example process 800 of processing user data files 610 with reference to a guideline document 630, in accordance with some embodiments. The medical record library 114M stores a time series of user data files 610 associated with a person 620 (e.g., a temporal sequence of doctor visit summaries and lab tests), and each user data file 610 is associated with a file date 810 on which the user data file 610 is created. In some embodiments, each user data file 610 may be mailed, faxed, or emailed to a computer system 600 (e.g., a server 106) associated with a medical request assessment application 625. Referring to FIG. 8A, in some embodiments, textual content may be extracted (operation 802``) from each user data file 610, e.g., using an optical character recognition (OCR) method. For example, the computer system 600 converts scanned images of the respective user data file 610 into machine-readable, editable textual content, allowing a user 140 who requested assessment of the user data file 610 to search, copy, edit, and highlight the textual content.

In some embodiments, the user data files 610 include unstructured data associated with medical records, and the computer system 600 implements (operation 804) a check under Health Insurance Portability and Accountability Act (HIPAA) to validate that all of the medical records are associated with the person 620, who is a target member of a service (e.g., a subscriber of a medical insurance service) provided by the application 625. The HIPAA is a federal law enacted in 1996 that sets national standards for protecting sensitive patient health information, ensuring the confidentiality, integrity, and availability of protected health information. In some embodiments, in accordance with a determination that of the medical records are not entirely validated under the HIPAA, the computer system 600 generates (operation 806) a HIPAA alert to remove a subset of the user data files 610 corresponding to one or more erroneous medical records. The one or more erroneous medical records may be validated and removed one by one, e.g., manually by the user 140. Remaining medical records are validated under the HIPAA. Conversely, in some embodiments, in accordance with a determination that the medical records are validated under the HIPAA, the computer system 600 processes (operation 808) unstructured data of the user data files 610 to extract the file dates 810 and generate content sections 812 of the user data files 610. In some embodiments, the computer system 600 includes a guideline picker module configured to determine (operation 814) a guideline document 630 based on the content sections 812 and/or the file dates 810 of the user data files 610.

Referring to FIG. 8B, in some embodiments, the computer system 600 supplements the user data files 610 with supplemental clinical information 816 that is not included in the user data files 610. Examples of the supplemental clinical information 816 includes, but is not limited to, electronic medical records 818 directly entered by medical practitioners via their user computing devices 116, 118, 120 and note information 820 provided by users 140 of the medical request assessment application 625. In some embodiments, after the guideline document 630 is determined (operation 814), the computer system 600 selects (operation 822) a guideline plan.

In some embodiments, based on the selected guideline plan, the guideline document 630 is preprocessed (operation 824), e.g., divided into a plurality of document chunks, and further converted (operation 826) into a plurality of embeddings 825 to be stored in a vector database, which is optionally part of the guideline library 114G. Further, in some embodiments, a custom embedding model 828 is trained using historic user data files or training guideline documents in a supervised manner, and applied to generate the plurality of embeddings 825 associated with the guideline document 630. More details on training of the custom embedding model 828 are discussed above with reference to FIG. 3. Alternatively, in some embodiments, a third-party embedding model 830 is applied to generate the plurality of embeddings 825 associated with the guideline document 630. The guideline document 630 is provided to a third-party server 106 that implements the third-party embedding model 830 and returns the plurality of embeddings 825. Additionally, in some embodiments, the medical request assessment application 625 includes an API configured to access the custom embedding model 828 or the third-party embedding model 830.

Further, in some embodiments, based on the selected guideline plan, the computer system 600 selects (operation 832) indications to determine a criterion indication plan 834, and may extract (operation 836) visual information from the user data files 610 (e.g., medical records) to include check boxes and/or photographs to be interpreted. Based on the criterion indication plan 838 and/or the plurality of embeddings 825, the computer system 600 determines a policy graph 840 including a decision tree of steps with dependencies. The policy graph 840 represents and controls a decision flow for a list of guideline policies 702. In some embodiments, a large language model (LLM) is applied (operation 842) to check node conditions and the policy graph 840 to determine the current state and what are the next decisions (nodes) to be made. The LLM may make decisions at a local level without reasoning for a corresponding task. Stated another way, in some embodiments, the policy graph 840 organizes one or more clinical criteria 602 and associated conditions 604 logically, and includes a plurality of decision nodes each corresponding to a criterion 602 or a condition 604. Each decision node is associated with one or more first prompts 606, which are processed by the LLM with reference to the user data files 610. The prompt results 710 and/or answers 608 are applied to determine the next decision nodes adaptively based on the policy graph 840. By these means, the LLM is applied to process a sequence of first prompts 606, while the first prompts 606 in the sequence are dynamically selected based on the prompt results 710 and the policy graph 840.

In an example, the plurality of first prompts 606 include a base prompt and a secondary prompt. In accordance with a policy graph 840, a secondary prompt is implemented based on the base prompt, and an answer to the secondary prompt is determined based on an answer to the base prompt.

Referring to FIG. 8C, in some embodiments, the user data files 610 are processed for information retrieval based on the file dates 810, the content sections 812, and the policy graph 840 (e.g., including criteria 602 and associated conditions 604). Filters may be applied (operation 844) on the content sections 812 of the user data files 610 to select a subset of the user data files 610. Examples of these filters include, but are not limited to, a date filter, a section filter, and a document type filter. Further, in some embodiments, a semantic search 846 is implemented on the selected subset of the user data files 610 based on the plurality of embeddings 825 stored in the vector database. In some embodiments, a keyword search 848 is implemented on the selected subset of the user data files 610. In some embodiments, a vision language model (VLM) 850 is applied to the selected subset of the user data files 610 to extract one or more text blocks 852, and the plurality of first prompts 606 are generated based on the one or more text blocks 852. Additionally, in some embodiments, resulting keywords of the semantic search 846 and the keyword search 848 are merged and ranked (operation 854) to generate target keywords 856. For instance, the resulting keywords may be fine-tuned using a cross encoder to generate the target keywords 856. Stated another way, in some embodiments, the computer system 600 extracts a plurality of preliminary keywords from the user data files 610 (e.g., via a keyword search 848), and applies a semantic analysis model to process the user data files 610 via a vector database to generate a set of semantic keywords (e.g., via a semantic search 846). For example, the plurality of preliminary keywords include one or more file dates 810 included in the user data files 610. The plurality of preliminary keywords and the set of semantic keywords are merged to generate (operation 854) an ordered list of target keywords. The plurality of first prompts 606 are generated based on the target keywords 856. In some embodiments, the ordered list of target keywords forms a structured data item 706 (FIG. 7) of the user data files 610.

In some embodiments, the updated keywords 856 and the text blocks 852 are applied to construct (operation 858) the first prompts 606 by denoting chunk with DCN or data citations and other configurable metadata. In some embodiments, the plurality of first prompts 606 include a target prompt 606T associated with one of the plurality of conditions 604 of the one or more criteria 602. The computer system 600 applies a function call 724 (FIG. 7) to process at least a structured data item 706 associated with the user data files 610 to generate a function result, and further generates the target prompt 606T based on the function result.

Further, in some embodiments, a generative model 650 is applied (operation 860) to execute the first prompts 606 determined based on the one or more criteria 602 and associated conditions 604. In some situations, the first prompts 606 may be finalized before the first prompts 606 are processed successively by the generative model 650. Alternatively, in some situations, a subset or all of the first prompts 606 may be selected and organized according to the policy graph 840 adaptively and in real time while the selected first prompts 606 are processed successively by the generative model 650. In some embodiments, the first prompts 606 may control a format of the prompt results 710, a combination of determination nodes of the policy graph 840, and/or a workflow associated with a user data file 610. For instance, a workflow may be split and consolidated successively to process a user data file having an extended length. In some embodiments, the generative model 650 includes an open source generative model or a third-party generative model. The medical request assessment application 625 includes a gateway 650A to access the open source generative model or a gateway 650B to access the third-party generative model.

In some embodiments, the prompt results 710 of the first prompts 606 are consolidated (operation 862) to generate the answers 608 to the criteria 602 and conditions 604 of the guideline policies 702. Further, in some embodiments, the computer system 600 applies the function call 724 (FIG. 7) to process both structured data item 706 and the prompt results 710 to generate a function result, and further generates a target prompt 606T based on the function result. In some embodiments, the function call 724 may be applied to process the prompt results 710 to generate the answers 608. The answers 608 correspond to decision nodes of the policy graph 840 representing the guideline policies 702 including one or more criteria 602 and associated conditions 604. The prompt results 710 and/or the answers 608 may be stored (operation 864) in a database.

Referring to FIG. 8D, in some embodiments, the computer system 600 determines (operation 866) that whether an end result 730 (FIG. 7)has been reached by applying the guideline policies 702 to the user data files 610 of the person 620 based on the policy graph 840 that logically connects the one or more criteria 602 and the associated conditions 604. In accordance with a determination that the end result 730 has not been reached, the computer system 600 continues to generate the first prompts 606 and process the first prompts 606 according to the decision nodes of the policy graph 840. In accordance with a determination that the end result 730 has been reached, the computer system 600 displays (operation 868) the end result 730 indicating whether the type 720 of medical requests is approved (operation for the person 620. In some embodiments, the end result 730 indicates that a medical request associated with the user data files 610 is approved, and the computer system 600 generates case decision note 870. Conversely, in some embodiments, when the end result 730 indicates that a medical request associated with the user data files 610 is denied, the user interface 640 may be updated (operation 874) to present evidence 872, e.g., jointly with the criteria 602 and associated conditions 604 of the guideline policies 702, allowing the user 140 to review a decision process associated with the policy graph 840. In some embodiments, a human-in-the-loop (HITL) model improvement process 876 is implemented to allow the computer system 600 to interact with the user 140 to update the process 700 or 800 of reviewing the user data files 610 with reference to the guideline document 630. Human expertise and feedback are integrated to improve generation of prompts 606, answers 608, aiming to enhance accuracy, reliability, and adaptability of the computer system 600. In other words, when the end result 730 indicates that a request is declined, the request may be forwarded to a user 140 to be manually reviewed, and when the end result 730 indicates that a request is approved, the request may not be forwarded to a user 140 to be manually reviewed any more.

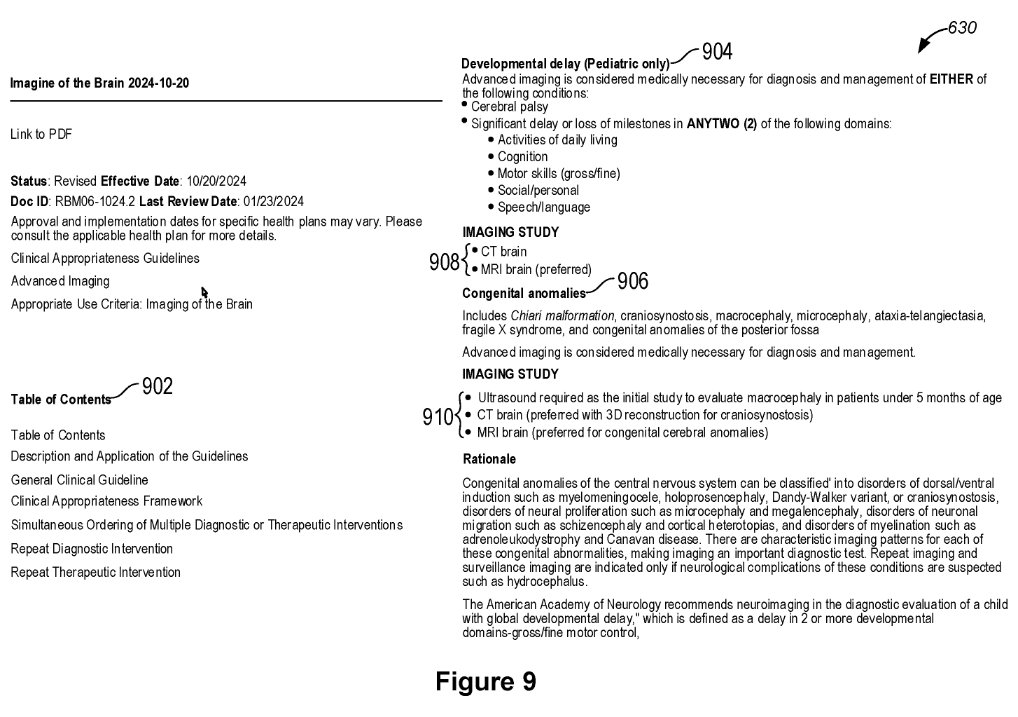

FIG. 9 illustrate excerpts of an example guideline document 630 followed to generate a plurality of prompts 606 for reviewing a braining imaging procedure, in accordance with some embodiments. The guideline document 630 corresponds to a type 720 of medical requests, and is configured to guide a process to review requests for approving a first medical procedure. The guideline document 630 indicates that the first medical procedure may be approved when each of a plurality of criteria 602 are satisfied. For example, when the first medical procedure includes a brain imaging procedure, the guideline document 630 has a table of contents 902 indicating that the guideline document 630 includes different subjects associated with the brain imaging procedure. Referring to FIG. 9B, in some embodiments, the brain imaging procedure may be approved for diagnosis of pediatric patents’ developmental delay 904 and congenital anomalies 906. Types 908 of the brain imaging procedure approvable for the developmental delay 904 include a CT brain scan and a magnetic resonance imaging (MRI) brain scan. . Types 910 of the brain imaging procedure approvable for the congenital anomalies 906 include an ultrasound scan, a CT brain scan, and an MRI brain scan. The guideline document 630 may further include description, rationales of using different types of brain imaging procedures, and related information of the developmental delay 904 and the congenital anomalies 906.

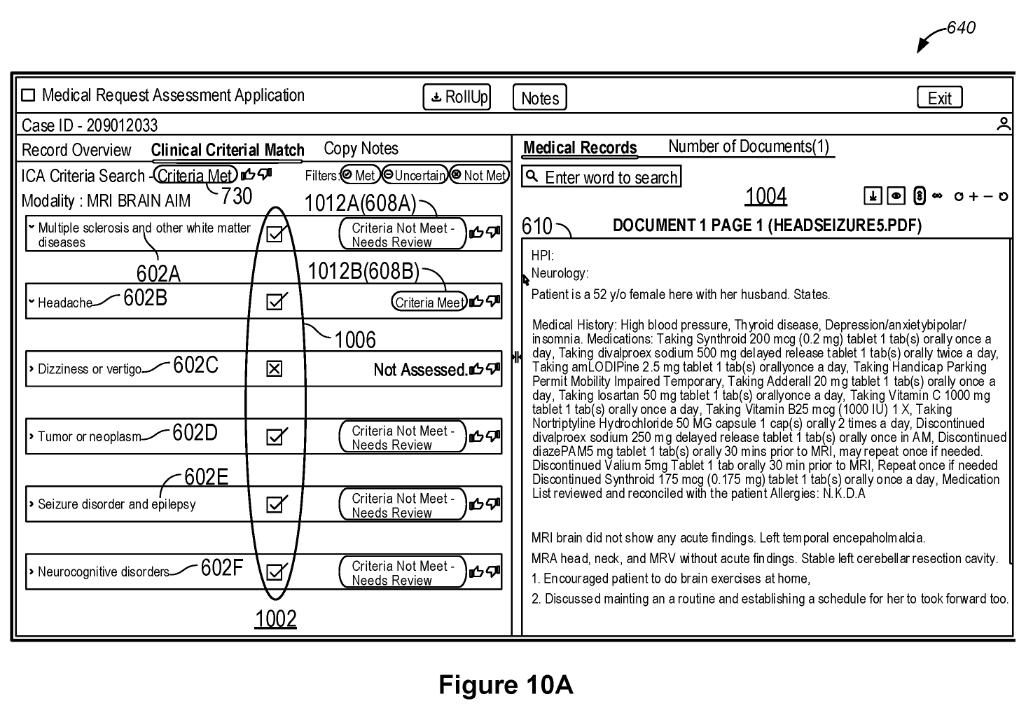

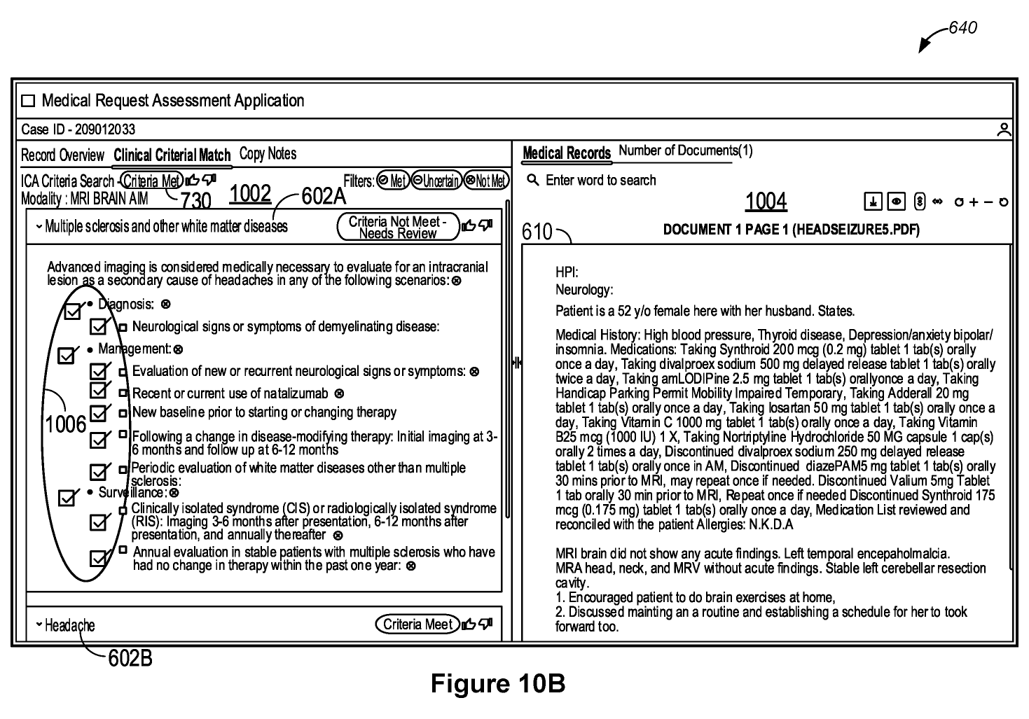

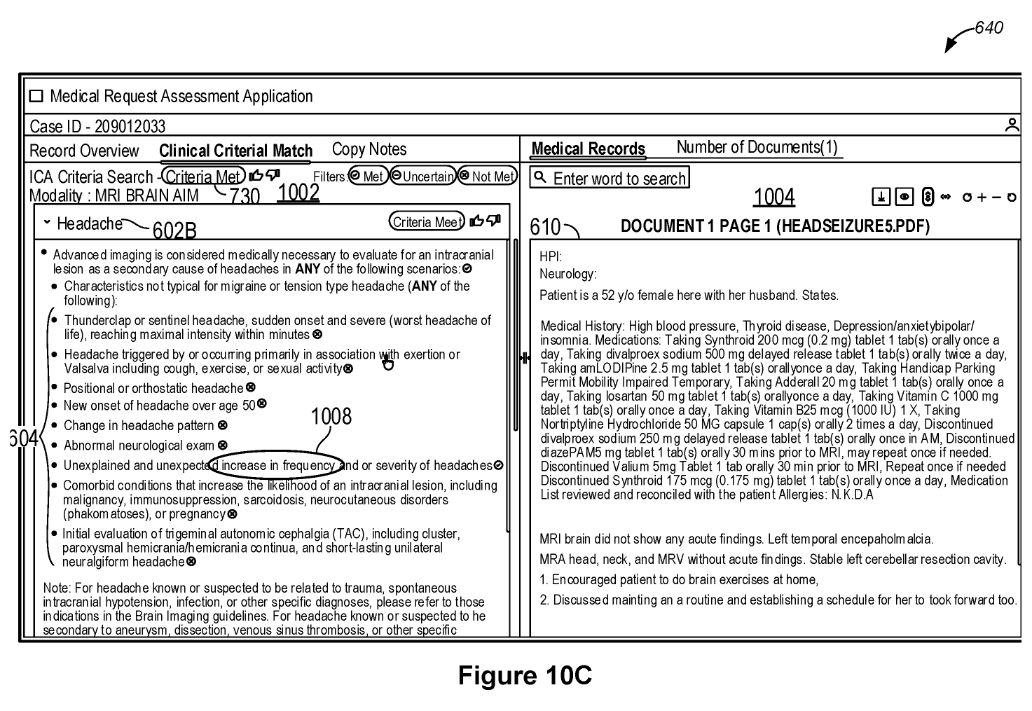

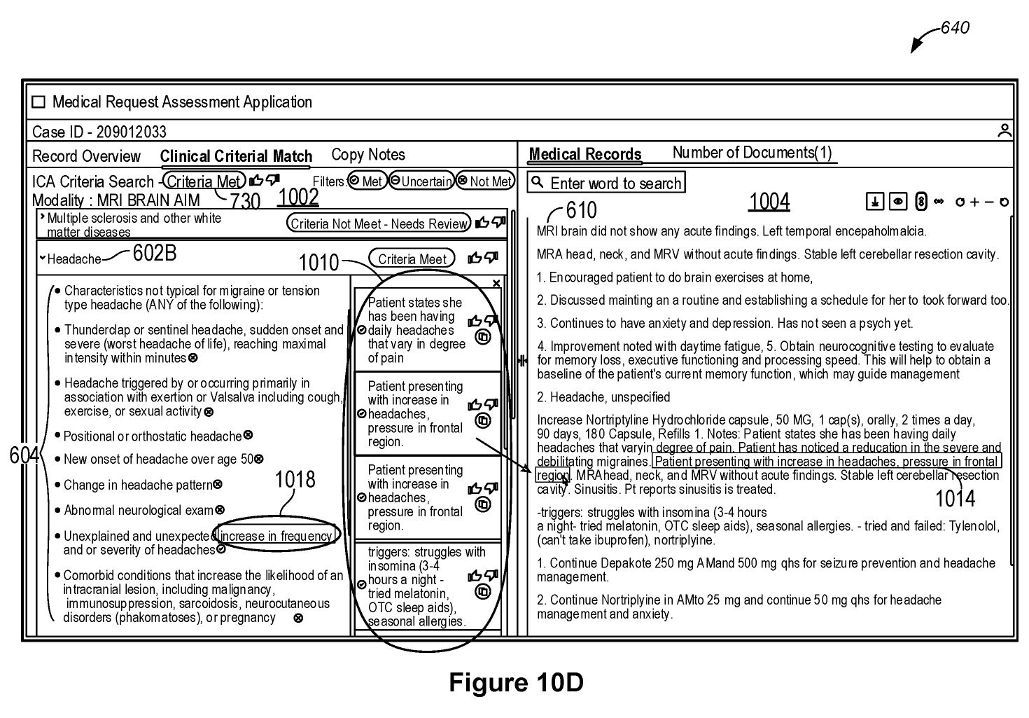

FIGS. 10A and 10B are example graphical user interfaces 640 rendered to display an ordered list of guideline policies 702 and a user data file 610 during execution of a medical request assessment application 625, in accordance with some embodiments. The computer system 600 obtains a guideline document 630 for assessing a type 720 of medical requests. The guideline document 630 is processed to identify one or more criteria 602 and the plurality of conditions 604 associated with each of the one or more criteria 602. The one or more criteria 602 and the plurality of conditions 604 are organized into the ordered list of guideline policies 702 and displayed on a user interface 640. For example, the user interface 640 includes a guideline policy panel 1002 for displaying a list of guideline policies 702 (e.g., criteria 602 and conditions 604) and an information panel 1004 on which a corresponding guideline document 630 or the user data files 610 are displayed for the user’s reference. Referring to FIG. 10A, the one or more clinical criteria 602 for approving an MRI brain scan include a criterion 602A for “multiple sclerosis and other white matter diseases,” a criterion 602B for “headache,” a criterion 602C for “dizziness or vertigo,” a criterion 602D for “tumor or neoplasm,” a criterion 602E for “seizure disorder and epilepsy,” and a criterion 602F for “ neurocognitive disorders.” The clinical criteria 602A-602F are listed on the guideline policy panel 1002 in a collapsed mode. In some embodiments (e.g., FIG. 10B), in response to a user selection on one of the clinical criteria 602A-602F, the selected one of the clinical criteria 602A-602F is expanded to display a plurality of conditions 604 associated with the selected one of the clinical criteria 602A-602F in an expanded mode. Alternatively, in some embodiments, all of the clinical criteria 602A-602F are expanded to display associated conditions 604 in the expanded mode.

In some embodiments, one or more user inputs 708 include a plurality of user selections of a subset of the plurality of conditions of the one or more criteria 602. In an example, each criterion 602 or each condition 604 corresponds to an actionable affordance item 1006, and is selected via a user input on the actionable affordance item 1006. The computer system determines that the selected subset of the plurality of conditions 604 of the one or more criteria 602 corresponds to a subset of first prompts, and the generative model 650 is applied to generate the plurality of prompt results 710 in response to the subset of first prompts. Referring to FIG. 10A, the criterion 602C is not selected for assessment, and its associated conditions 604 are not assessed based on the user data files 610 displayed on the information panel 1004. In some situations, the first prompts 606 corresponding to the criterion 602C may not be generated. Alternatively, in some situations, the first prompts 606 are preexisting and not used by the generative model 650. Conversely, the criteria 602A, 602B, and 602D-602F are selected, and their associated conditions 604 are assessed based on a subset of first prompts 606 corresponding to the criteria 602A, 602B, and 602D-602F.

Referring to FIG. 10A, in some embodiments, a plurality of answers 608 to the selected criteria 602A, 602B, and 602D-602F are displayed in the collapsed mode, when the criteria 602 are displayed without their associated conditions 604. For example, the criteria 602A and 602D-602F correspond to a first visual item 1012A of a first answer 608A, e.g., “Criteria Not Met – Needs Review,” and the criterion 602B corresponds to a second visual item 1012B of a second answer 608B, e.g., “Criteria Met.” Referring to FIG. 10B, in some embodiments, a plurality of answers 608 to the selected criteria 602A, 602B, and 602D-602F are displayed in the collapsed mode, when the criteria 602 are displayed without their associated conditions 604. For example, the criteria 602A and 602D-602F correspond to a first answer 608A, represented by a first visual item 1012A displaying a message of “Criteria Not Met – Needs Review.” The criterion 602B corresponds to a second answer 608B, represented by a second visual item 1012B displaying a message of “Criteria Met.”

In some embodiments, each of the plurality of answers 608 is binary (e.g., has a value of “Criteria Not Met – Needs Review” or “Criteria Met”). For a first condition, in accordance with a determination that the first condition is not satisfied by the user data files 610, the computer system 600 displaying a first visual item 1012A immediately adjacent to a first guideline policy 702 (e.g., corresponding to the criterion 602A) representing the first condition. For a second condition, in accordance with a determination that the second condition is not satisfied by the user data files, displaying a second visual item 1012B immediately adjacent to a second guideline policy 702 (e.g., corresponding to the criterion 602B) representing the second condition. The second visual item 1012B is different from the first visual item 1012A. In an example not shown, the first visual item 1012A includes a box that is not checked, and the second visual item 1012B includes a box that is checked. In another example, the first visual item 1012A includes a box that is unfilled, and or the second visual item 1012B includes a box that is filled.

Alternatively, in some embodiments not shown, for a first condition, in accordance with a determination that the first condition is not satisfied by the user data files 610, the computer system 600 displays a first guideline policy representing the first condition with a first visual effect. For a second conditions, in accordance with a determination that the second condition is satisfied by the user data files, the computer system 600 displays the respective second condition with a second visual effect (e.g., in bold, in a distinct color, in a larger font size). The second visual effect is different from the first visual effect.

Referring to FIG. 10B, in some embodiments, a plurality of answers 608 to individual conditions 604 of the selected criterion 602A are displayed in the expanded mode, when the criterion 602A are displayed with their associated conditions 604 that grouped under three groups, “Diagnosis,” “Management,” and “Surveillance.” In accordance with the policy graph 840, the criterion 602A is satisfied if any of the three groups of conditions (“Diagnosis,” “Management,” and “Surveillance”) is satisfied. In this example, a group of conditions (e.g., “Surveillance”) may include a plurality of conditions 604, and is satisfied only if all of its conditions 604 are satisfied. The user 140 may manually disable a group of conditions or a particular condition by entering a user input 708 on a respective affordance item 1006.

In some embodiments, the computer system 600 determines an end result 730 of applying the guideline policies 702 (e.g., criteria 602 and conditions 604) to the user data files 610 of the person 620 based on a policy graph 840 logically connecting the one or more criteria 602 and the associated conditions 604, and displays the end result 730 indicating whether the type 720 of medical requests is approved for the person 620. Further, in some embodiments not shown, in accordance with a determination that the end result 730 indicates that the type of medical requests is not approved for the person 620, the computer system 600 generates an instruction to review the user data files 610 for the type 720 of medical requests.

FIGS. 10C and 10D are example graphical user interfaces 640 rendered to display a criterion 602B that is satisfied by a user data file 610, in accordance with some embodiments. In accordance with the policy graph 840, the criterion 602B is satisfied if any of nine conditions 604 (e.g., “unexplained and unexpected increase in frequency and/or severity of headaches”) is satisfied. Referring to FIG. 10C, the condition 604 or its keyword 1008 that is satisfied is highlighted, e.g., in a different color or font. Referring to FIG. 10D, in some embodiments, a rationale for satisfying a condition is explained by listing a plurality of relevant statements 1010 identified from the user data files 610. In response to a user selection of one of the plurality of relevant statements 1010, a corresponding portion of the user data files 610 including the selected relevant statement 1010 is displayed in the information panel, allowing the user 140 to review context of the selected relevant statement 1010.

More specifically, in some embodiments, referring to FIG. 10D, a keyword 1018 (e.g., “increase in frequencies … of headaches) is highlighted in one of the plurality of conditions 604 of a target criterion 602B. The computer system 600 displays, on the user interface 640, the portion of the user data files concurrently with displaying a subset of the ordered list of guideline policies 702. An evidence sentence 1014 corresponding to the keyword 1008 is visually highlighted in the portion of the user data files 610.

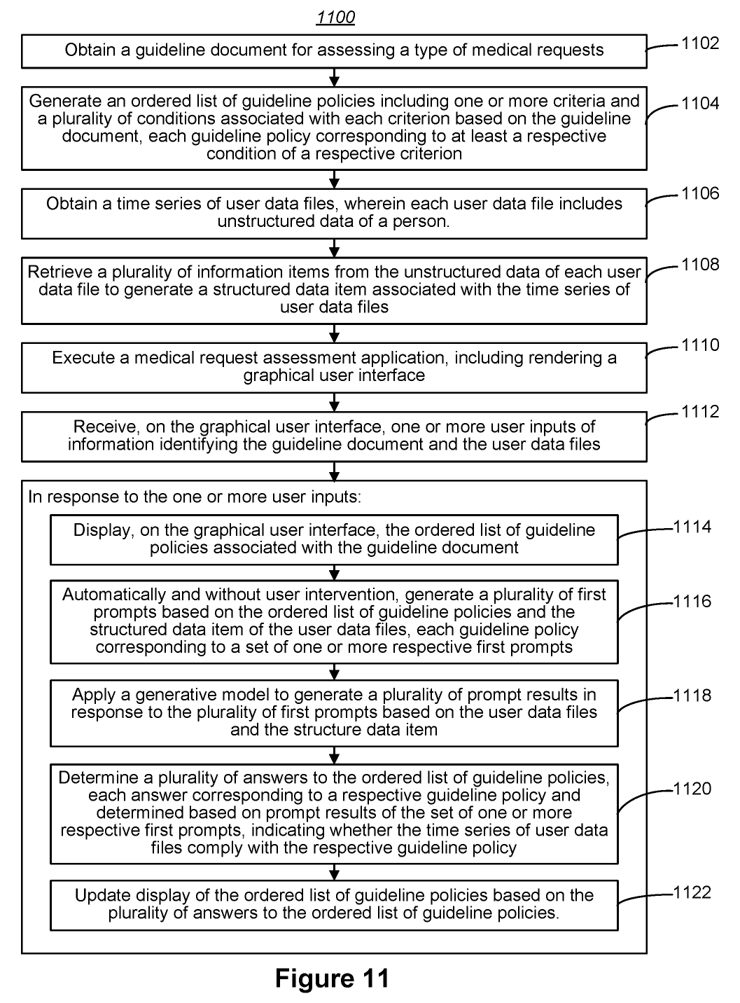

FIG. 11 is a flow diagram of an example method 1100 for reviewing a time series of user data files 610 with reference to a guideline document 630, in accordance with some embodiments. Method 1100 is, optionally, governed by instructions that are stored in a non-transitory computer readable storage medium and that are executed by one or more processors of the system. Each of the operations shown in FIG. 11 may correspond to instructions stored in a computer memory or non-transitory computer readable storage medium. The computer readable storage medium may include a magnetic or optical disk storage device, solid state storage devices such as Flash memory, or other non-volatile memory device or devices. The instructions stored on the computer readable storage medium may include one or more of: source code, assembly language code, object code, or other instruction format that is interpreted by one or more processors. Some operations in method 1100 may be combined and/or the order of some operations may be changed.

The method 1100 is implemented at a computer system having one or more processors and memory for storing one or more instructions. The computer system 600 obtains (operation 1102) a guideline document 630 for assessing a type 720 of medical requests and generates (operation 1104) an ordered list of guideline policies 702 including one or more criteria 602 and a plurality of conditions 604 associated with each criterion 602 based on the guideline document 630. Each guideline policy 702 corresponds to at least a respective condition 604 of a respective criterion. The computer system 600 obtains (operation 1106) a time series of user data files 610, and each user data file 310 includes unstructured data of a person 620 (e.g., a patient). The computer system 600 retrieves (operation 1108) a plurality of information items from the unstructured data of each user data file 310 to generate a structured data item 706 associated with the time series of user data files 610.

The computer system 600 executes (operation 1110) a medical request assessment application, including rendering a graphical user interface 640, and receives (operation 1112), on the graphical user interface 640, one or more user inputs 708 of information identifying the guideline document 630 and the user data files 610. In response to the one or more user inputs 708, the computer system 600 displays (operation 1114), on the graphical user interface 640, the ordered list of guideline policies 702 associated with the guideline document 630. Automatically and without user intervention, the computer system 600 generates (operation 1116) a plurality of first prompts 606 based on the ordered list of guideline policies 702 and the structured data item 706 of the user data files 610, and each guideline policy 702 corresponds to a set of one or more respective first prompts 606. The computer system 600 applies (operation 1118) a generative model 650 to generate a plurality of prompt results 710 in response to the plurality of first prompts 606 based on the user data files 610 and the structure data item 706, determines (operation 1120) a plurality of answers 608 to the ordered list of guideline policies 702. Each answer 608 corresponds to a respective guideline policy 702 and is determined based on prompt results 710 of the set of one or more respective first prompts 606, indicating whether the time series of user data files 610 comply with the respective guideline policy 702. The computer system 600 updates (operation 1122) display of the ordered list of guideline policies 702 based on the plurality of answers 608 to the ordered list of guideline policies 702.

In some embodiments, after obtaining the guideline document 630 for assessing the type 720 of medical requests, the computer system 600 processes the guideline document 630 to identify the one or more criteria 602 and the plurality of conditions 604 associated with each of the one or more criteria 602, and organizes the one or more criteria 602 and the plurality of conditions 604 into the ordered list of guideline policies 702.

In some embodiments, the computer system 600 stores, in a guideline library, the ordered list of guideline policies 702. The ordered list of guideline policies 702 is extracted from the guideline library in response to the one or more user inputs 708. Further, in some embodiments, the computer system 600 generates the plurality of first prompts 606 by extracting, from the guideline library, a plurality of preliminary prompts 714 generated based on the guideline document 630 and modifying the plurality of preliminary prompts 714 based on the structured data item 706 of the user data files 610 to generate the plurality of first prompts 606.

In some embodiments, after obtaining the user data files 610 of the person 620, the computer system 600 applies a function call 716 to process a subset of information items 704 and generate a supplemental information item 718 and consolidates the plurality of information items 704 and the supplemental information item 718 to generate the structured data item 706 associated with the user data files 610.

In some embodiments, the computer system 600 stores, in a medical record library 114M, the user data files 610 and the structured data item 706 associated with the person 620. The user data files 610 and the structured data item 706 associated with the person 620 are extracted from the medical record library 114M in response to the one or more user inputs 708.

In some embodiments, the one or more user inputs 708 include a first user selection of the type 720 of medical requests. In response to the first user selection, the computer system 600 identifies the guideline document 630 for assessing the type 720 of medical requests, and extracts, from a guideline library, the ordered list of guideline policies 702.

In some embodiments, the one or more user inputs 708 include a second user selection of an identifier of the person 620. I response to the second user selection, the computer system 600 extracts, from a medical record library 114M, the user data files 610 and the structured data item 706 of the person 620. The plurality of first prompts 606 are generated based on the user data files 610 and the structured data item 706, and the generative model 650 is applied to process the first prompts 606 to generate the plurality of prompt results 710.

Further, in some embodiments, the plurality of first prompts 606 include a target prompt 606T associated with one of the plurality of conditions 604 of the one or more criteria 602. The computer system 600 generates the plurality of first prompts 606 by applying a function call 724 to at least the structured data item 706 to generate a function result and generating the target prompt 606T based on the function result.

In some embodiments, each of the plurality of answers 608 is binary. For a first condition, in accordance with a determination that the first condition 604 is not satisfied by the user data files 610, the computer system 600 displays a first visual item 1012A (FIG. 10A) immediately adjacent to a first guideline policy 702 representing the first condition 608A. For a second condition, in accordance with a determination that the second condition 604 is satisfied by the user data files 610, the computer system 600 displays a second visual item 1012B (FIG. 10A) immediately adjacent to a second guideline policy 702 representing the second condition, wherein the second visual item 1012B is different from the first visual item 1012A.

In some embodiments, each of the plurality of answers 608 is binary. For a first condition, in accordance with a determination that the first condition 604 is not satisfied by the user data files 610, the computer system 600 displays a first guideline policy 702 representing the first condition 604 with a first visual effect. For a second condition, in accordance with a determination that the second condition 604 is satisfied by the user data files 610, the computer system 600 displays the respective second condition 604 with a second visual effect, wherein the second visual effect is different from the first visual effect.

In some embodiments, the computer system 600 displays updates display of the ordered list of guideline policies 702 by determining an end result 730 (FIGS. 10A-10D) of applying the guideline policies 702 to the user data files 610 of the person 620 based on a policy graph 840 logically connecting the one or more criteria 602 and the associated conditions 604 and/or by displaying the end result 730 indicating whether the type 720 of medical requests is approved for the person 620. Further, in some embodiments, in accordance with a determination that the end result 730 indicates that the type 720 of medical requests is not approved for the person 620, the computer system 600 generates an instruction to review the user data files 610 for the type 720 of medical requests.

In some embodiments, the computer system 600 highlights a keyword 1008 in one of the plurality of conditions 604 of a target criterion, displays, on the user interface 640, a portion of the user data files 610 concurrently with displaying the ordered list of guideline policies 702, and visually highlights an evidence sentence in the portion of the user data files 610. The evidence sentence corresponds to the keyword 1008.

In some embodiments, the one or more user inputs 708 include a plurality of user selections of a subset of the plurality of conditions 604 of the one or more criteria 602. The computer system 600 determines that the subset of the plurality of conditions 604 of the one or more criteria 602 corresponds to a subset of first prompts 606, wherein the generative model 650 is applied to generate the plurality of prompt results 710 in response to the subset of first prompts 606.

In some embodiments, the computer system 600 extracts a plurality of preliminary keywords from the user data files 610, applies a semantic analysis model to process the user data files 610 via a vector database to generate a set of semantic keywords, and merges the plurality of preliminary keywords and the set of semantic keywords to generate an ordered list of target keywords. The plurality of first prompts 606 are generated based on the target keywords. Further, in some embodiments, the plurality of preliminary keywords include one or more dates included in the user data files 610.

In some embodiments, a vision language model 850 is applied to the user data files 610 to extract one or more text blocks 852, and the plurality of first prompts 606 are generated based on the one or more text blocks 852.

In some embodiments, the type 720 of medical requests corresponds to requests for approving a first medical procedure, and the guideline document 630 indicates that the first medical procedure may be approved when each of a plurality of criteria 602 are satisfied.

In some embodiments, the plurality of first prompts 606 include a base prompt and a secondary prompt. In accordance with a policy graph 840, the computer system 600 determines an answer to the secondary prompt based on an answer to the base prompt.

It should be understood that the particular order in which the operations in FIG. 11 have been described are merely exemplary and are not intended to indicate that the described order is the only order in which the operations could be performed. One of ordinary skill in the art would recognize various ways to reorder the operations described herein. Additionally, it should be noted that details of other processes described herein with respect to FIGS. 1-10D are also applicable in an analogous manner to method 1100 described above with respect to FIG. 11. For brevity, these details are not repeated here.

Memory is also used to store instructions and data associated with the method 1100, and includes high-speed random access memory, such as DRAM, SRAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory, optionally, includes one or more storage devices remotely located from one or more processing units. Memory, or alternatively the non-volatile memory within memory, includes a non-transitory computer readable storage medium. In some embodiments, memory, or the non-transitory computer readable storage medium of memory, stores the programs, modules, and data structures, or a subset or superset for implementing method 1100.

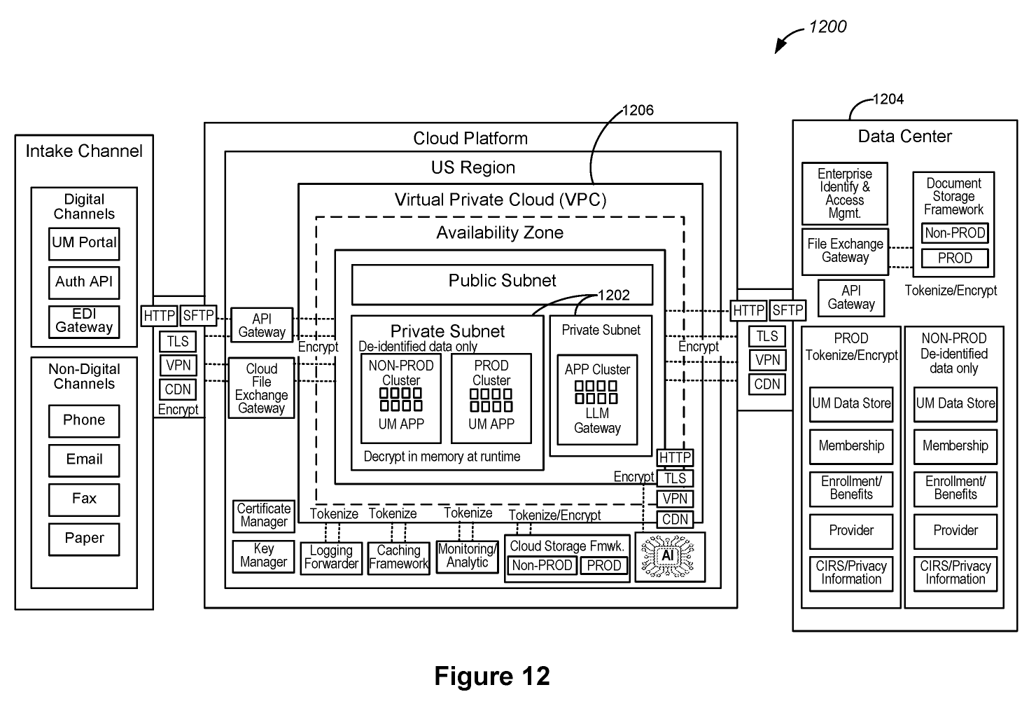

FIG. 12 is a block diagram of an example network environment 1200 configured to manage patient health information (PHI), in accordance with some embodiments. The network environment 1200 is implemented based on a hybrid cloud and on-premise resources. The PHI is ingested, processed, and stored with separation of different portions of the environment 1200, which complies with the Health Insurance Portability and Accountability Act (HIPAA) of 1996. The HIPAA is a US federal law designed to protect sensitive PHI from disclosure without consent, improve health insurance portability, and reduce healthcare fraud. It mandates strict national standards for safeguarding medical records and electronic data. For example, the network environment 1200 satisfies requirements of data residency, encryption boundaries, and transmission pathways under HIPAA §164.312(e).

In some embodiments, during data ingestion, the PHI enters through secure, encrypted channels (e.g., Transport Layer Security (TLS) 1.2 or higher) and is received by privately-controlled ingress points within an on-premises and virtual private cloud network 1202. In some embodiments, the PHI is processed within private data centers 1204 and approved virtual private cloud (VPC) 1206. Workloads in the cloud platform 1208 are logically isolated and use Applicant-managed encryption keys. In some embodiments, the PHI is stored within a managed infrastructure, which enforces data tokenization (e.g., using Secure Hash Algorithm 256-bit (SHA-256)) and envelop level encryption at rest (e.g., using Advanced Encryption Standard with a 256-bit key(AES-256)) and in transit. Cloud data stores are region-restricted to comply with one or more U.S. data residency requirements.

In some embodiments, different portions of the environment 1200 are separated. for example, production, staging, and development portions are separated via one or more of logical and network segmentation, access controls, data handling. Each environment portion may operate in a separate logically separated cluster within VPC 1206, with no shared credentials or data access. Role-based access controls (RBAC) and multi-factor authentication may be applied consistently. The PHI is never replicated to non-production environments, and synthetic or de- identified data is used for testing.

Some implementations are directed to verification of security and compliance. Encryption boundaries may be managed through key management service, with distinct key sets for on-premises and cloud resources. For transmission pathways, all data transfers may use encrypted protocols (HTTPS/TLS or SFTP with AES 256 encryption) with an approve Public Key Infrastructure (PKI). for monitoring and auditing, continuous logging, security information and event management (SIEM) monitoring, and quarterly compliance reviews may validate adherence to HIPAA §164.312(e).

In some embodiments, an artificial intelligence (AI) solution currently leverages technology components from third parties (e.g., Distyl, Amazon Web Services (AWS), and OpenAI) to enable data analysis, decision support, and automation within one or more utilization management processes. The AI solution complies with U.S.-only data handling restrictions, and operates within the bounds of applicable law, confidentiality obligations, and private information security standards. The third-party technology components may form part of a broader AI infrastructure and be updated or replaced over time to maintain. In some embodiments, an AI solution is included within a private information security program, and configured to address access control, audit logging, encryption, system integrity, and session management.

Some implementations are directed bias and fairness testing. In some embodiments, tests are implemented in the network environment 1200 based on age and gender attributes. This narrow scope may not demonstrate adequate assurance against unintended bias for a certain medical population. In some embodiments, a disparate impact ratio review lacks statistical thresholds or confidence intervals to confirm meaningful bias detection.

In some embodiments, a bias test is implemented with available, complete demographic data (e.g., age, gender, race, zip code, income, national origin, ethnicity, language, marital status). The bias test is aligned with the National Institute of Science and Technology (NIST) AI Risk Management Framework that provides a structured approach to managing risk, including bias. In some situations, if any of these demographic attributes is either incomplete or unavailable, it increases the potential for adverse impacts. A demographic attribute data is considered incomplete when available for less than 80 % of the population. Alternatively, in some embodiments, an official bias methodology, which is used in courts and by agencies like the Equal Employment Opportunity Commission (EEOC), is used in the environment 1200. This act as a statistical tool to flag situations where there appears to be a disproportionate negative effect on a protected group.

In some embodiments, the environment 1200 is configured to manage performance drift, retraining, and postimplementation monitoring for compliance tracking. A precision of an UM AI solution may be at least a target threshold (e.g., 90 %), and the UM AI solution may perform at or above an existing human reviewer performance. In some embodiments, a team of clinicians and technical experts, including medical directors, is embedded in the post-implementation audit process, and provides oversight on the use of AI for clinical decision making. Additionally, a quality review team expands to audit, test, and monitor outputs of the UM AI Solution. In some embodiments, performance results are measured to confirm appropriate medical necessity interpretation by the UM AI solution, and remain within the oversight of a clinical team. In some situations, for each clinical guideline that has been implemented, the team audits a statistically significant number of all approved cases. If the model performs below a precision target, the AI solution may perform root cause analysis and remediation. In some embodiments, during any remediation phase, the UM AI solution may be used for touchless approvals. Conversely, in some embodiments, during any remediation phase, the UM AI solution may not be used for touchless approvals.

Although the subject matter has been described in terms of exemplary embodiments, it is not limited thereto. Rather, the appended claims should be construed broadly, to include other variants and embodiments, which may be made by those skilled in the art.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Additionally, it will be understood that, although the terms “first,” “second,” etc. may be used herein to describe various elements, these elements should not be limited by these terms. These terms are only used to distinguish one element from another.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

Although various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages can be implemented in hardware, firmware, software or any combination thereof.

What is claimed is:

1. A computer implemented method, comprising:
at a computer system having one or more processors and memory for storing one or more instructions:
(1) obtaining a guideline document for assessing a type of medical requests;
generating an ordered list of guideline policies including one or more criteria and a plurality of conditions associated with each criterion based on the guideline document, each guideline policy corresponding to at least a respective condition of a respective criterion;
(2) obtaining a time series of user data files, wherein each user data file includes unstructured data of a person;
retrieving a plurality of information items from the unstructured data of each user data file to generate a structured data item associated with the time series of user data files;
(3) executing a medical request assessment application, including rendering a graphical user interface;
receiving, on the graphical user interface, one or more user inputs of information identifying the guideline document and the user data files ; and
in response to the one or more user inputs:
displaying, on the graphical user interface, the ordered list of guideline policies associated with the guideline document;
automatically and without user intervention, generating a plurality of first prompts based on the ordered list of guideline policies and the structured data item of the user data files, each guideline policy corresponding to a set of one or more respective first prompts;
applying a generative model to generate a plurality of prompt results in response to the plurality of first prompts based on the user data files and the structure data item;
determining a plurality of answers to the ordered list of guideline policies, each answer corresponding to a respective guideline policy and determined based on prompt results of the set of one or more respective first prompts, indicating whether the time series of user data files comply with the respective guideline policy; and
updating display of the ordered list of guideline policies based on the plurality of answers to the ordered list of guideline policies.

2. The method of claim 1 further comprising, after obtaining the guideline document for assessing the type of medical requests:
processing the guideline document to identify the one or more criteria and the plurality of conditions associated with each of the one or more criteria; and organizing the one or more criteria and the plurality of conditions into the ordered list of guideline policies.

3. The method of claim 1 further comprising:
storing, in a guideline library, the ordered list of guideline policies, wherein the ordered list of guideline policies is extracted from the guideline library in response to the one or more user inputs.

4. The method of claim 3, generating the plurality of first prompts further comprising:
extracting, from the guideline library, a plurality of preliminary prompts generated based on the guideline document; and
modifying the plurality of preliminary prompts based on the structured data item of the user data files to generate the plurality of first prompts.

5. The method of claim 1 further comprising, after obtaining the user data files of the person:
applying a function call to process a subset of information items and generate a supplemental information item; and
consolidating the plurality of information items and the supplemental information item to generate the structured data item associated with the user data files.

6. The method of claim 1 further comprising:
storing, in a medical record library, the user data files and the structured data item associated with the person; wherein the user data files and the structured data item associated with the person are extracted from the medical record library in response to the one or more user inputs.

7. The method of claim 1 wherein the one or more user inputs include a first user selection of the type of medical requests, the method further comprising, in response to the first user selection:
identifying the guideline document for assessing the type of medical requests; and
extracting, from a guideline library, the ordered list of guideline policies.

8. The method of claim 1 wherein the one or more user inputs include receiving a second user selection of an identifier of the person, the method further comprising, in response to the second user selection:
extracting, from a medical record library, the user data files and the structured data item of the person;
wherein the plurality of first prompts are generated based on the user data files and the structured data item, and the generative model is applied to process the plurality of first prompts to generate the plurality of prompt results.

9. The method of claim 8, wherein the plurality of first prompts include a target prompt associated with one of the plurality of conditions of the one or more criteria, and generating the plurality of first prompts further comprising:
applying a function call to at least the structured data item to generate a function result; and
generating the target prompt based on the function result.

10. The method of claim 1 wherein each of the plurality of answers is binary, and updating display of the ordered list of guideline policies further comprises:
for a first condition, in accordance with a determination that the first condition is not satisfied by the user data files, displaying a first visual item immediately adjacent to a first guideline policy representing the first condition; and
for a second condition, in accordance with a determination that the second condition is satisfied by the user data files, displaying a second visual item immediately adjacent to a second guideline policy representing the second condition, wherein the second visual item is different from the first visual item.

11. The method of claim 1 wherein each of the plurality of answers is binary, and updating display of the ordered list of guideline policies further comprises:
for a first condition, in accordance with a determination that the first condition is not satisfied by the user data files, displaying a first guideline policy representing the first condition with a first visual effect; and
for a second condition, in accordance with a determination that the second condition is satisfied by the user data files, displaying the respective second condition with a second visual effect, wherein the second visual effect is different from the first visual effect.

12. The method of claim 1 wherein updating display of the ordered list of guideline policies further comprises one or more of:
determining an end result of applying the guideline policies to the user data files of the person based on a policy graph logically connecting the one or more criteria and respective conditions; and
displaying the end result indicating whether the type of medical requests is approved for the person; and
wherein the method further comprises, in accordance with a determination that the end result indicates that the type of medical requests is not approved for the person, generating an instruction to review the user data files for the type of medical requests.

13. The method of claim 1 further comprising:
highlighting a keyword in one of the plurality of conditions of a target criterion; and
displaying, on the graphical user interface, a portion of the user data files concurrently with displaying the ordered list of guideline policies; and
visually highlighting an evidence sentence in the portion of the user data files, the evidence sentence corresponding to the keyword.

14. The method of claim 1, wherein the one or more user inputs include a plurality of user selections of a subset of the plurality of conditions of the one or more criteria, the method further comprising:
determining that the subset of the plurality of conditions of the one or more criteria corresponds to a subset of first prompts, wherein the generative model is applied to generate the plurality of prompt results in response to the subset of first prompts.

15. The method of claim 1 further comprising:
extracting a plurality of preliminary keywords from the user data files; and
applying a semantic analysis model to process the user data files via a vector database to generate a set of semantic keywords; and
merging the plurality of preliminary keywords and the set of semantic keywords to generate an ordered list of target keywords;
wherein the plurality of first prompts are generated based on the target keywords; and
wherein the plurality of preliminary keywords include one or more dates included in the user data files.

16. The method of claim 1 further comprising:
applying a vision language model to the user data files to extract one or more text blocks, wherein the plurality of first prompts are generated based on the one or more text blocks.

17. The method of claim 1 wherein the type of medical requests corresponds to requests for approving a first medical procedure, and the guideline document indicates that the first medical procedure may be approved when each of a plurality of criteria are satisfied.

18. The method of claim 1 wherein the plurality of first prompts include a base prompt and a secondary prompt, the method further comprising:
in accordance with a policy graph, determining an answer to the secondary prompt based on an answer to the base prompt.

19. A computer system, comprising:
one or more processors; and
memory having instructions stored thereon, which when executed by the one or more processors cause the processors to perform operations for:
(1) obtaining a guideline document for assessing a type of medical requests;
generating an ordered list of guideline policies including one or more criteria and a plurality of conditions associated with each criterion based on the guideline document, each guideline policy corresponding to at least a respective condition of a respective criterion;
(2) obtaining a time series of user data files, wherein each user data file includes unstructured data of a person;
retrieving a plurality of information items from the unstructured data of each user data file to generate a structured data item associated with the time series of user data files;
(3) executing a medical request assessment application, including rendering a graphical user interface;
receiving, on the graphical user interface, one or more user inputs of information identifying the guideline document and the user data files ; and
in response to the one or more user inputs:
displaying, on the graphical user interface, the ordered list of guideline policies associated with the guideline document;
automatically and without user intervention, generating a plurality of first prompts based on the ordered list of guideline policies and the structured data item of the user data files, each guideline policy corresponding to a set of one or more respective first prompts;
applying a generative model to generate a plurality of prompt results in response to the plurality of first prompts based on the user data files and the structure data item;
determining a plurality of answers to the ordered list of guideline policies, each answer corresponding to a respective guideline policy and determined based on prompt results of the set of one or more respective first prompts, indicating whether the time series of user data files comply with the respective guideline policy; and
updating display of the ordered list of guideline policies based on the plurality of answers to the ordered list of guideline policies.

20. A non-transitory computer-readable storage medium, having instructions stored thereon, which when executed by one or more processors of a server system cause the processors to perform operations for:
(1) obtaining a guideline document for assessing a type of medical requests; generating an ordered list of guideline policies including one or more criteria and a plurality of conditions associated with each criterion based on the guideline document, each guideline policy corresponding to at least a respective condition of a respective criterion;
(2) obtaining a time series of user data files, wherein each user data file includes unstructured data of a person;
retrieving a plurality of information items from the unstructured data of each user data file to generate a structured data item associated with the time series of user data files;
(3) executing a medical request assessment application, including rendering a graphical user interface;
receiving, on the graphical user interface, one or more user inputs of information identifying the guideline document and the user data files; and
in response to the one or more user inputs:
displaying, on the graphical user interface, the ordered list of guideline policies associated with the guideline document;
automatically and without user intervention, generating a plurality of first prompts based on the ordered list of guideline policies and the structured data item of the user data files, each guideline policy corresponding to a set of one or more respective first prompts;
applying a generative model to generate a plurality of prompt results in response to the plurality of first prompts based on the user data files and the structure data item;
determining a plurality of answers to the ordered list of guideline policies, each answer corresponding to a respective guideline policy and determined based on prompt results of the set of one or more respective first prompts, indicating whether the time series of user data files comply with the respective guideline policy; and
updating display of the ordered list of guideline policies based on the plurality of answers to the ordered list of guideline policies.
